(19) United States
(12) Patent Application Publication
(10) Pub. No.: US 2022/0079762 A1
(43) Pub. Date: Mar. 17, 2022

(54) Anchor Delivery System and Associated Methods

(71) Applicant: Bluesail New Valve Technology Asia Ltd., Kowloon (HK)

(72) Inventors: Eugene Serina, Fremont, CA (US); Minh Nguyen, Midway City, CA (US); Sherrie Yang, Redondo Beach, CA (US)

(73) Assignee: Bluesail New Valve Technology Asia Ltd., Kowloon (HK)

(21) Appl. No.: 17/475,089

(22) Filed: Sep. 14, 2021

Related U.S. Application Data
(60) Provisional application No. 63/077,846, filed on Sep. 14, 2020.

(51) Int. Cl. A61F 2/24; A61M 25/01

(52) U.S. Cl. CPC A61F 2/2466; A61M 25/10; A61F 2220/0008; A61M 25/0147

(57) ABSTRACT

Anchor deployment systems and methods, particularly for deployment of annuloplasty implant systems in a catheter-based procedure, are provided herein. Such systems can include a double-basket structure having expandable centering structure and outer anchor support frame disposed over at least over a proximal portion of the centering structure. The structures can be advanced together as an assembly. Subsequent expansion of the centering structure centers the assembly within the valve annulus while the anchor support frame positions the anchors at suitable positions around the valve annulus. The centering member can be contracted and withdrawn into the catheter to allow normal valve function, while the anchors remain supported by the anchor support frame for deployment by the clinician and implantation into surrounding tissue. The delivery catheter can include a proximal handle with various control features including selectors to allow selective implantation of all or any combination of anchors concurrently.

### CROSS-REFERENCE TO RELATED APPLICATIONS

The present application claims the benefit of priority to U.S. Provisional Application No. 63/077,846, filed Sep. 14, 2020, the contents of which are hereby incorporated by reference in their entirety for all purposes.

The present application is generally related to co-pending and co-owned application Ser. No. ______ [Atty Docket No. 107360-1263240-000110US] filed concurrently herewith, the contents of which are hereby incorporated by reference in their entirety for all purposes.

### BACKGROUND

Treatments for heart valve deficiencies, in particular mitral valve regurgitation, are widely varied. Mitral valve regurgitation is a condition that occurs when the mitral valve annulus is dilated or misshapen such that there is insufficient coaptation between the posterior mitral leaflet (PML) and the anterior mitral leaflet (AML), which allows blood to flow backward from the left ventricle (LV) into the left atrium of the heart (see heart anatomy in FIG. 1). Over time, this deficiency worsens and can lead to congestive heart failure, atrial fibrillation, pulmonary hypertension and ultimately death. Among the earliest approaches to mitral valve repair is the prosthetic annuloplasty ring developed in 1968. The prosthetic aimed to reform the proper shape of the valve annulus to provide proper leaflet coaptation so that normal valve function was restored. As compared to earlier approaches, the prosthetic annuloplasty ring to remodel the shape of the valve annulus has provided consistent and reliably positive patient outcomes and long-lasting results. One major drawback of this early approach, however, is that the annuloplasty ring is manually sutured into place around the valve annulus so that the implantation required an open-heart surgical procedure, which present considerable risks and challenges, particularly for patients already in poor health. In recent decades, a number of catheter-based approaches have been developed that attempt to similarly remodel the shape of the valve annulus while avoiding the risks associated with an open-heart surgical procedure. These catheter-based approaches include a variety of approaches, including cinching implants, leaflet clips, as well as sutures and splints that span across a heart cavity. However, few if any approaches thus far have provided the consistency and reliability in implantation and patient outcomes as the original prosthetic annuloplasty ring approach noted above. In addition, as with many catheter based procedures, precise placement and implantation is more challenging due to the enclosed environment and limited visualization. Accordingly, these catheter-based procedures can be tedious and time-consuming, with the outcome of the procedure often heavily reliant on the skill of the physician. While more recent developments have sought to replicate the advantages of a prosthetic annuloplasty ring within a catheter-based approach, as of yet, these approaches have so far failed to replicate the success of a convention surgically implanted annuloplasty ring, due largely to the complexities in anchoring before securing the annuloplasty ring. Thus, there is need for a catheter-based approach that allows for improved ease and consistency in positioning and implanting of anchors.

### BRIEF SUMMARY

The present disclosure relates to anchor delivery systems, in particular, for anchoring of annuloplasty implant systems, and methods of anchor delivery and deployment. While the systems and methods are described in regard to treatment of the mitral valve, it is appreciated that these concepts can be applicable to any heart valve and any implant anchored in the body.

In one aspect, the invention pertains to an anchor delivery system for delivery anchors for a heart valve implant, such as an annuloplasty ring. The delivery system can include: a delivery catheter configured to extend from outside the patient to within the heart of a patient; multiple anchors disposed within a distal portion of the delivery catheter; multiple torque wires releasably coupled to the respective shafts of the anchors; and a proximal handle of the catheter that controls actuation of the torque wires during anchor delivery. In some embodiments, each anchor includes: a shaft extending between proximal and distal ends; a distal penetrating tip disposed at the distal end; a lock mechanism disposed along the shaft at or near the proximal end; and a couple-release mechanism disposed along the shaft at or near the proximal end and configured for decoupling a torque wire coupled with the shaft. In some embodiments, each lock mechanism and couple-release mechanism are configured so that actuation of the lock mechanism with the implant effects actuation of the release mechanism thereby decoupling the respective shaft and torque wire. In some embodiments, the couple-release mechanism is located proximally of the lock mechanism on the anchor shaft. The lock mechanism can include a resilient ramped surface that tapers toward the anchor shaft in a proximal direction, wherein the ramped surface extends from an inside of a collar disposed on the shaft. In some embodiments, the lock feature includes one or more hypotubes, each having a proximal tapered portion to facilitate passage of a tabbed collar thereon and a flat distal facing surface that abuts against the inwardly extending tabs of the collar to lock the implant. The lock mechanism can include a series of such hypotubes along the shaft so as to be adjustable. The couple-release mechanism can include a protruding feature that engages a corresponding protruding feature at or near a distal end of the torque wire, thereby coupling the torque wire with the shaft when the corresponding features are engaged and releasing the torque wire when the features are disengaged. In some embodiments, the anchor release mechanism includes a longitudinally translatable core wire extending through the torque wire that, when present, the core wire forces a locking component outward to engage a slot in an outer tube of the anchor. Retraction of the core wire allows the locking component to resiliently deflect inward, thereby disengaging from the slot of the outer tube to detach the torque tube from the anchor. In other embodiments, the release mechanism can include a rotating cam lock that is rotatable between a locked position in an outer sleeve and an unlocked position in which the cam lock can be withdrawn from the sleeve. In some embodiments, the locking mechanism of the implant can include a hook coupling that extends through a hole in the anchor when the ring is advanced, thereby locking the ring to the anchor. In other embodiments, the ring locking mechanism can include a ball-detent coupling in which a spring-loaded ball extends from a collar of the ring and through a hole or detent in the anchor, thereby locking the ring to the anchor. It is appreciated that various other configurations and connection mechanisms could be used. In some embodiments, the couple-release mechanism is configured so that the engaged protruding features disengage when the resilient ramped surface of the lock mechanism is engaged with the ring, thereby decoupling the torque wire.

In another aspect, the anchor delivery catheter includes a proximal handle that includes one or more torque driving mechanisms configured to torque each torque wire to facilitate driving of the screw anchors into tissue surrounding the valve annulus. The handle can include a manually rotatable actuator to engage the torque wires with the torque mechanisms. The proximal handle can also include selector features for each torque wire to allow a user to select any, all or any combination of torque wires to allow selective driving of any, all or any combination of anchors into tissue. In some embodiments, the proximal handle is configured to allow any torque wire to be selected and driven in reverse to allow removal of one or more selected implanted anchors.

In yet another aspect, the anchor delivery system can include an expandable anchor support supporting multiple anchors to facilitate positioning of the anchors about the valve annulus; and an expandable centering member disposed at least partly within the expandable support frame. The expandable anchor support can include multiple guide tubes that support the anchors at distal ends thereof, the torque wires extending through the guide tubes to allow driving of the anchors into tissue while supported by the guide tubes. Typically, the guide tubes splay laterally outward along distal portions thereof by an expandable scaffold of the anchor support during anchor deployment. The expandable anchor can be configured to support the anchors spaced laterally outward from the expandable centering member to avoid interference between the anchors and the centering member. In some embodiments, the expandable anchor support is configured to support the anchors spaced radially with a uniform spacing. In other embodiments, the expandable support frame is configured to support the anchors spaced radially in a non-uniform spacing corresponding to the morphology of the valve annulus. The expandable anchor support can further include includes spring portions along the guide tubes proximal of the anchors to allows the anchor support and anchors to be conformable during delivery and allows for uniform anchor and tissue interaction before deployment. The expandable anchor support can also be defined as a flexible band that extends circumferentially about the longitudinal axis and that is supported by the spring portions so that the band accommodates varying approach angles as the anchors are advanced against the annulus. In some embodiments, the centering member is an expandable scaffold or basket that allows flow of blood therethrough. In other embodiments, the centering structure can be a balloon, either a standard type intravascular balloon or a perfusion balloon to allow blood flow therethrough. In one aspect, the centering member is independently expandable and axial movable relative the anchor support. The centering member can be defined in various shapes to improve apposition within the annulus, including a shape with an enlarged flattened region that can be created by use of a series of hypotubes, an offset enlarged region to control deployment on one side of the annulus, or an enlarged region with a depression to receive the annulus therein.

In another aspect, the invention pertains to a method of delivering a plurality of anchors for a valve implant. Exemplary methods can include steps of: advancing a delivery catheter through vasculature to a heart chamber adjacent the valve annulus, wherein the delivery catheter includes multiple anchors disposed in distal portions thereof, the anchors being supported on an expandable anchor support in a constrained configuration within the distal portion of the catheter; advancing an expandable centering member through the valve annulus, the anchor support being disposed over a proximal portion of the centering member and the centering member being separable from the anchor support; expanding the centering member within the valve annulus to center the centering member and anchor support within the valve annulus, thereby positioning the anchors along tissue surrounding the valve annulus; contracting the expandable centering member while the support frame remains supporting the anchors about the valve annulus; and driving at least some of the anchors into the surrounding tissue concurrently. In some embodiments, the centering member is axially movable and/or expandable independently from the support structure. In such embodiments, the method can further include retracting the expandable centering member at least partly into the delivery catheter after contracting to allow valve function during driving of the anchors. Expanding the centering member can include foreshortening the centering member by axial movement of a central shaft of the centering member via an actuation control on the proximal handle.

In some such methods, each anchor includes a shaft extending between proximal and distal ends and a distal penetrating screw tip disposed at the distal end and multiple torque wires releasably coupled to the respective shafts of the anchors at or near the proximal ends of the shaft, the torque wires extending to a proximal handle of the delivery catheter. In such embodiments, driving some or all of the anchors into the surrounding tissue can include manually rotating an actuator control on the proximal handle to engage one or more torquing mechanisms with the respective torque wires of the respective anchors.

In some embodiments, the methods can further include selecting any, all or any combination of anchors by manually adjusting selectable control features of the proximal handle, wherein the selectable control features engage or disengage the torque mechanisms from the respective torque wires. The method may also entail assessing implantation of the anchors by visualization techniques. After determining that one or more anchors are not satisfactorily placed, the physician may select the one or more anchors based on the assessment and reverse actuation of one or more corresponding torque wires to explant the one or more selected anchors; and remove the anchors entirely or reposition the explanted anchors and subsequently drive the respective anchors into tissue as described previously. In some embodiments, the methods can further include removing the delivery catheter while leaving the torque wires coupled with the anchors to facilitate subsequent deployment of an implant over the torque wires, and subsequently advancing a valve implant over the torque wires and securing the valve implant to the anchors implanted in the tissue surrounding the valve annulus, thereby reforming the valve annulus.

### BRIEF DESCRIPTION OF THE DRAWINGS

FIG. 1A shows a cross-sectional side view of an implanted annuloplasty implant system, in accordance with some embodiments of the invention.

FIG. 1B shows the anatomy of the mitral valve.

FIGS. 2A-2D show a conventional prosthetic annuloplasty ring implanted in an open-heart surgical procedure.

FIG. 3A shows an anchor delivery catheter in accordance with some embodiments.

FIG. 3B shows a distal anchor delivery portion of the anchor delivery catheter in accordance with some embodiments.

FIG. 3C shows a proximal control handle of the anchor delivery catheter in accordance with some embodiments.

FIGS. 4A-4B show a side view and rear view of an expandable anchor support structure in the expanded deployed configuration in accordance with some embodiments.

FIG. 4C shows a side view of an expandable anchor support with an expanded centering member disposed within during anchor deployment in accordance with some embodiments.

FIG. 4D shows a side view of an expandable anchor support with an alternative design of the expanded centering member disposed within during anchor deployment in accordance with some embodiments.

FIGS. 5A-5C show several views of a screw anchor in accordance with some embodiments.

FIGS. 6A-6B show a torque wire and anchor coupled and decoupled by a torque wire couple-release mechanism, respectively, in accordance with some embodiments.

FIGS. 7A-7D show cross-sectional views of the torque-wire couple-release mechanism of the embodiment of FIGS. 6A-8B.

FIGS. 8 and 9A-9B show an alternative coupling-release mechanism having a rotatable cam lock in accordance with some embodiments.

FIG. 10 shows an adjustable ring locking feature for securing the ring to the anchors in accordance with some embodiments.

FIGS. 11A-11B show alternative ring locking features. FIG. 11A shows a ring locking feature having a hook coupling for securing the ring to the anchors in accordance with some embodiments. FIG. 11B shows s ring locking feature having a ball-detent coupling for securing the ring to the anchors in accordance with some embodiments.

FIGS. 12A-12D show several views of an annuloplasty ring design in accordance with some embodiments.

FIGS. 13A-14B show an adjustable annuloplasty ring design in accordance with some embodiments.

FIG. 15 shows an alternative annuloplasty ring design sliding on multiple cables in accordance with some embodiments.

FIGS. 16A and 16B show the annuloplasty ring of FIG. 15 in a delivery configuration and a deployed implantation configuration, respectively, in accordance with some embodiments.

FIG. 17 shows an exemplary annuloplasty implant system implanted on a model of a mitral valve annulus in accordance with some embodiments.

FIGS. 18A-18B show views of an annuloplasty ring being deployed from an annuloplasty ring delivery catheter in accordance with some embodiments.

FIGS. 19A-19C show several views of an annuloplasty ring delivery catheter in accordance with some embodiments.

FIG. 20 shows an articulable access sheath that can be advanced intravascularly to an atrium of the heart, such as in a transfemoral approach, to provide access for the respective delivery catheters of the anchors and annuloplasty ring in accordance with some embodiments.

FIG. 21 shows the access sheath advanced and penetrating through the septal wall and into the left atrium to provide access to mitral valve in the left atrium.

FIGS. 22A-22H show sequential views of delivery and implantation of the annuloplasty implant system in accordance with some embodiments

FIGS. 23A-23D show alternate centering structure designs in accordance with some embodiments.

FIG. 24 show a centering structure with a flattened region formed by hypotubes in accordance with some embodiments.

FIGS. 25 and 26A-26E show an anchor support band that can accommodate varying approach angles in accordance with some embodiments.

FIGS. 27 and 28A-28C show another anchor support with slidable anchors that can accommodate varying approach angles in accordance with some embodiments.

FIGS. 29A-29C show alternative anchor coupling-release mechanisms in accordance with some embodiments.

FIGS. 30A-30D show alternative anchor coupling-release mechanisms in accordance with some embodiments.

### DESCRIPTION OF THE INVENTION

The present invention pertains to an anchor delivery system, delivery catheters and methods of anchor delivery, particularly for use with an annuloplasty implant system that seeks to provide similar reliability and consistency in patient outcomes as a conventional prosthetic annuloplasty ring implanted in an open-heart surgical procedure. Advantageously, the invention allows for a similar approach but within a minimally invasive catheter-based approach. In one aspect, the system separates deployment of the anchors from deployment of the annuloplasty ring, thereby allowing the physician greater focus on proper anchor placement and implantation before implantation of the annuloplasty ring. The invention further allows for improved ease of use and time efficiency by allowing the physician to implant multiple anchors simultaneously, while still allowing for independent anchor deployment as needed to ensure optimal placement of all anchors. While the system and methods described herein pertain to anchors for a particular annuloplasty implant system utilizing an improved 3D annuloplasty ring, it is appreciated that the anchor deployment catheter and methods can be used with a variety of different types of annuloplasty rings, including two-dimensional (2D) annuloplasty rings, and implant systems.

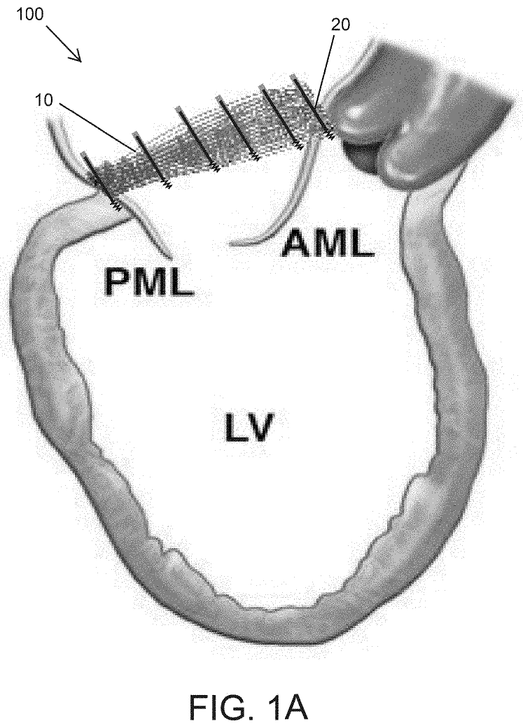

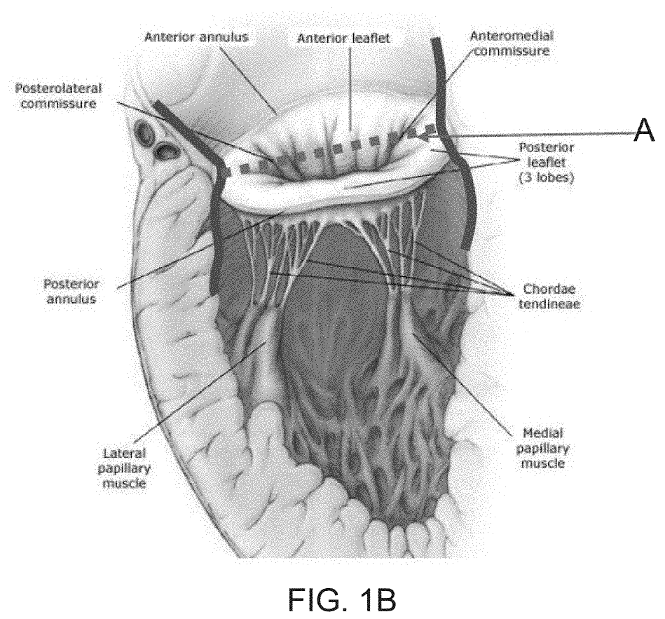

FIG. 1A shows a cross-sectional side view of an exemplary annuloplasty implant system 100 in accordance with some embodiments. The implant system includes multiple screw anchors 20 that are implanted in tissue surrounding the mitral valve annulus. The anchors are implanted at positions distributed evenly about the valve annulus of the mitral valve (see FIG. 1A). In some embodiments, the anchors are distributed unevenly, for example at location where more anchoring forces are needed due to the morphology of the valve. Typically, between 5-20 anchors are used, typically within a range of 6 to 12, preferably about 8 anchors although any suitable number of anchors can be used. A 3D annuloplasty ring 10 is disposed adjacent the valve annulus and securely locked to the anchors by a ring locking mechanism, thereby reforming the shape of the valve annulus. The annuloplasty ring 10 can be specially configured to reform the 3D shape of the valve annulus to improve coaptation of the AML and PML leaflets and restore normal valve function. The means by which the implant system is delivered and implanted is described in detail below. FIG. 1B shows the anatomy of the mitral valve and in particular the location of the annulus A relative the atrium above the annulus and the ventricle below the annulus.

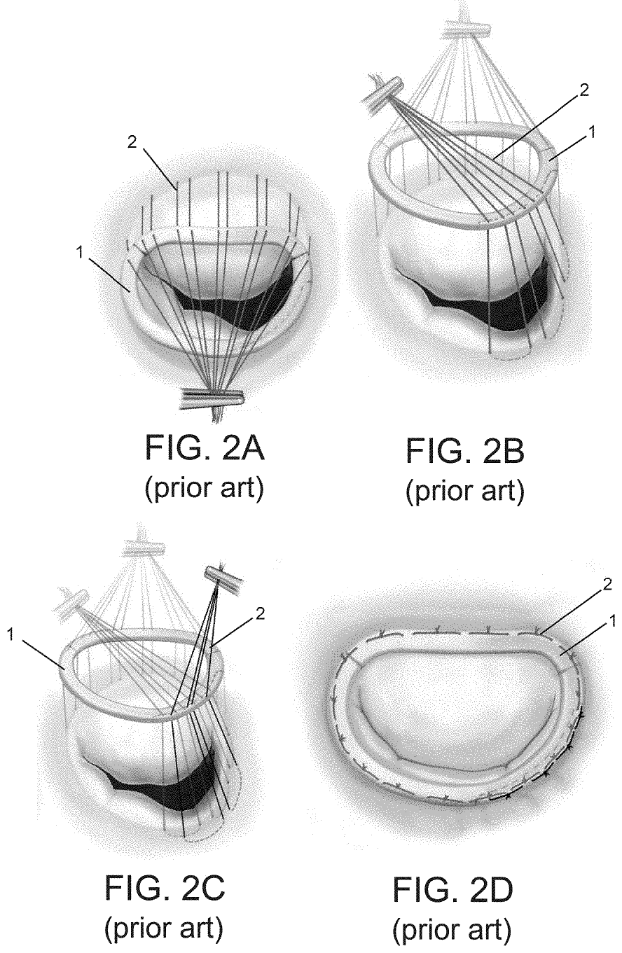

FIGS. 2A-2D show a conventional annuloplasty ring implantation in an open-heart surgical procedure. This conventional procedure is often considered the gold standard in surgical of mitral regurgitation repair and involves implantation of a semi-rigid annuloplasty ring 1 around the valve annulus. As shown in FIG. 2A, sutures 2 are implanted along the valve annulus, spaced precisely around the valve annulus. The sutures 2 are then sewn through the smaller sized annuloplasty ring 1, as shown in FIG. 2B. As shown, the spacing of the sutures is smaller on the ring. The ring is then pushed down upon the annulus, as shown in FIG. 2C, drawing the dilated valve annulus to the smaller diameter of the annuloplasty ring. The sutured are then tied off completing the repair, as shown in FIG. 2D. As noted above, this approach has provided reliably consistent results, yet suffers the considerable drawbacks associated with manually suturing tissues in an open-heart surgical procedure.

In one aspect, the annuloplasty implant system of FIG. 1A is designed to replicate the conventional annuloplasty ring surgical procedure, depicted in FIGS. 2A-2D, in order to provide similar consistency and reliability in patient outcomes. Advantageously, the concepts described herein allow this procedure to be performed in a catheter-based approach (e.g. a transfemoral catheter approach) that avoids the drawback and risks associated with an open-heart surgical procedure. In one aspect, the implantation method of the annuloplasty implant system described herein involves two main steps: (i) delivering and deploying multiple anchors with cables; and (ii) delivering an annuloplasty ring over the cables to secure with the anchors. Separating anchor deployment from ring deployment allows for greater design focus on improving ease and consistency in positioning and implanting the anchors around the valve annulus. In another aspect, this approach allows for use of an improved annuloplasty ring design having a 3D shape that remodels the valve annulus to a more anatomically correct shape and leads to better clinical performance. Conventional annuloplasty rings typically have a 2D shape (e.g. flat), which neglect the contours and morphology of the patient's natural valve annulus. Utilizing a 3D shape allows for an annuloplasty ring that can not only conform to the patient's morphology, but can also reform the overall shape and contours of the valve annulus to a desired 3D shape, rather than just reducing the diameter to a 2D shape. In some embodiments, this improved annuloplasty design can be customized specifically for a patient's anatomy to reform the valve annulus to the desired form.

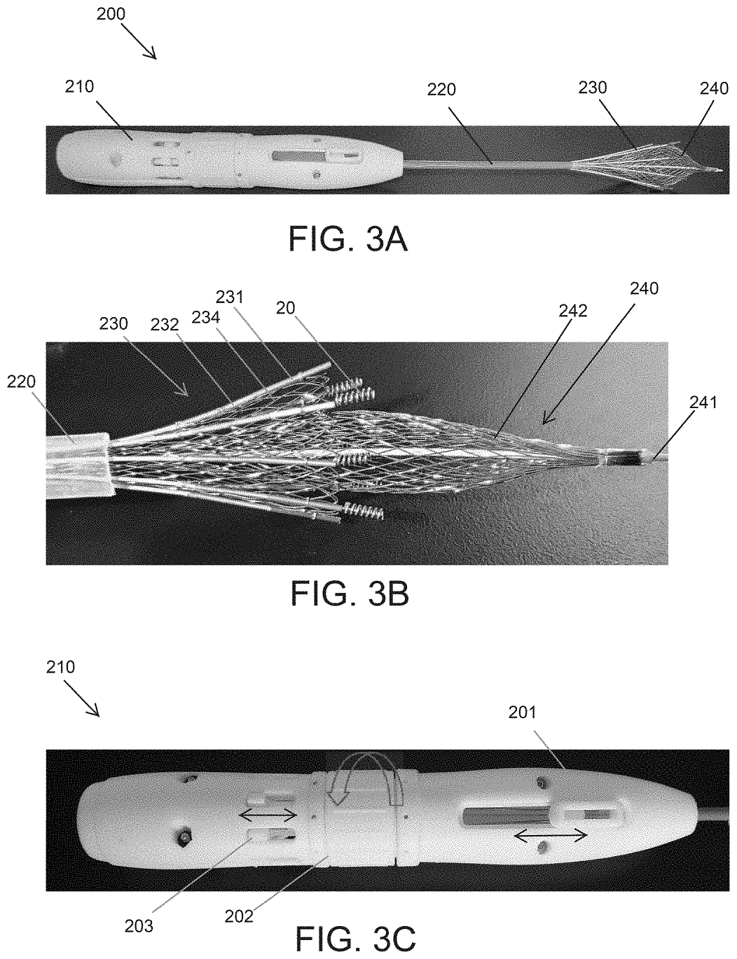

FIG. 3A shows an anchor delivery catheter 200 in accordance with some embodiments. Anchor delivery catheter 200 includes a proximal handle 210, an elongate flexible shaft 220, and an expandable anchor support 230 and expandable centering member 240 that are advanceable from the distal end. In some embodiments, the anchor support 230 and centering member 240 are each expandable frames, scaffolds or baskets, the anchor support 230 being an outer basket and the centering member 240 being an inner basket such that expansion of the inner basket expands the outer basket. In some embodiments, the centering member is a balloon, however, in this embodiment, the centering member is a scaffold or basket, which is advantageous as it allows blood to circulate while the centering member is expanded. In addition, the centering member is separable from the anchor support such that the centering member can be contracted while the anchor support remains expanded, which allows the valve to function while the anchors are adjusted and/or driven into the tissue. This also allows the physician to spend more time to accurately position and reliably deploy the anchors, as compared to systems where centering structures are integral with the anchor deployment mechanism.

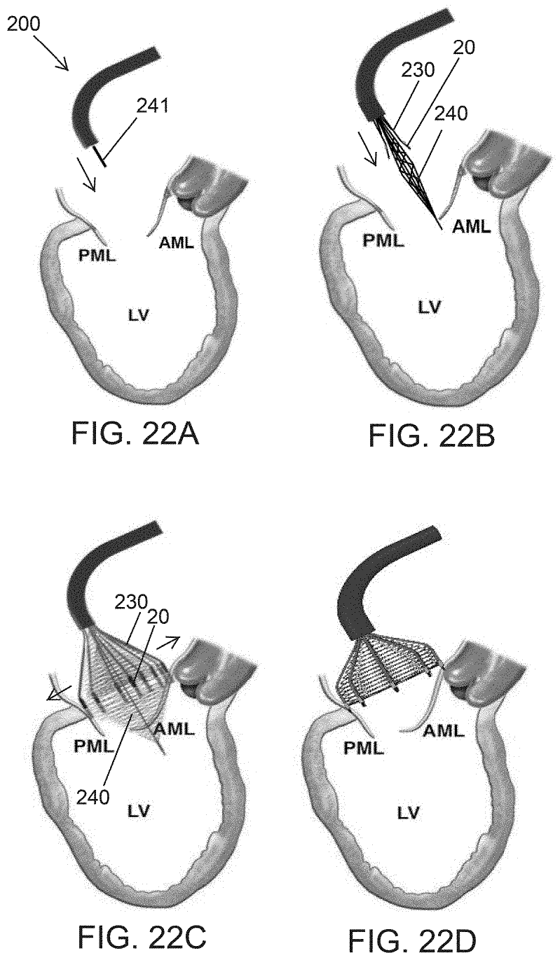

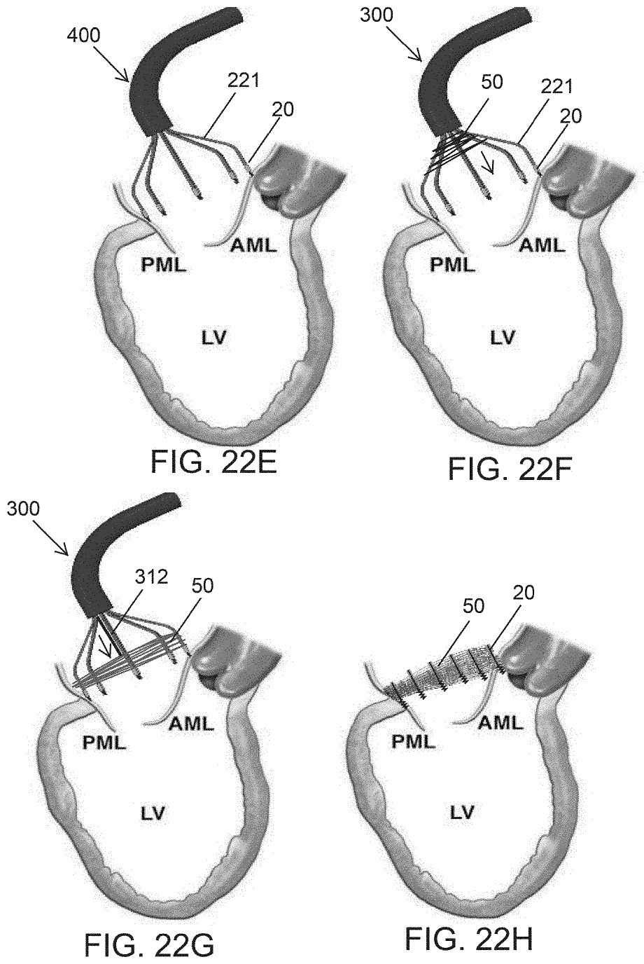

FIG. 3B shows a detail view of the distal portion of the anchor delivery catheter 200. The anchor support 230 includes support guides 231 with torque wires (not visible) therein. Multiple screw anchors 20 are releasably coupled to the distal ends of the torque wires and extend distally of the support guides 231. In some embodiments, the catheter includes between six and twelve anchors, preferably about eight anchors, disposed radially about the anchor support. Torquing of the individual torque wires, by torque mechanisms that are disposed within the handle, drives each anchor 20 into the tissue after positioning of the anchors about the valve annulus. The support guides 231 are evenly spaced and may be interconnected by an expandable struts, mesh or frame 234 extending between the support guides. The distal portion of the support guides 231 splay outward so that the distal anchors are spaced apart from the centering member, which avoids interference between the anchors and centering basket during anchor delivery. The distal portion of the support guides 230 also include a spring portion 232, which allows the anchor support frame and anchors to be more conformable during delivery and allows for more uniform anchor and tissue interaction before deployment. The centering member 240 includes a central shaft 241 to which is attached an expandable mesh or basket 242 that when foreshortened expands laterally outward. For example, axial movement of the central shaft from the proximal handle expands and contracts the centering member 240 to facilitate centering during anchor delivery. As discussed in more detail in FIGS. 22A-22D, the anchor support 230 and centering member 240 are advanced from the distal end of catheter 200, the centering member is expanded, thereby centering the assembly within the valve annulus and also expanding the anchor support thereon to position the anchors about the valve annulus. Further advancement engages the anchors with the tissue surrounding the valve annulus, after which the centering member can be contracted and withdrawn to allow blood flow while the anchors are implanted into the tissue.

FIG. 3C shows a proximal control handle 210 of the anchor delivery catheter and includes control features for controlling delivery and deployment of the anchors. Centering switch 201 effects axial linear motion for opening and closing of the centering basket 240. Torque actuator 202 engages torque mechanisms that torque the individual torque wires for rotational deployment or removal of anchors. Rotation of torque actuator 202 in one direction (e.g. clockwise) effect clockwise rotation of engaged torque wires to screw anchors into tissue, while rotation of the torque actuator 202 in the opposite direction effects counter-clockwise rotation of engage torque wires to effect removal of anchors. This feature allows for simultaneous deployment of all screw anchors 20. Selector switches 203 allows the physician to select one or more individual anchors to apply torque for removing one or more anchors, after which the physician can adjust or reattempt deployment on an individual basis. As shown, moving the switch 203 in one direction engages the torque wire with the torque mechanism such that rotation of actuator 2 effects torquing of the respective torque wire, while moving the switch in the opposite direction disengages the torque wire from the torque mechanism such that the respective torque wire is not torqued when the actuator 2 is rotated. This feature allows a physician to select any, all or any combination of anchors for deployment. However, if the position of a single anchor is then determined to be suboptimal by visualization techniques, an individual anchor can be selected and removed, repositioned as needed, then subsequently redeployed into the tissue.

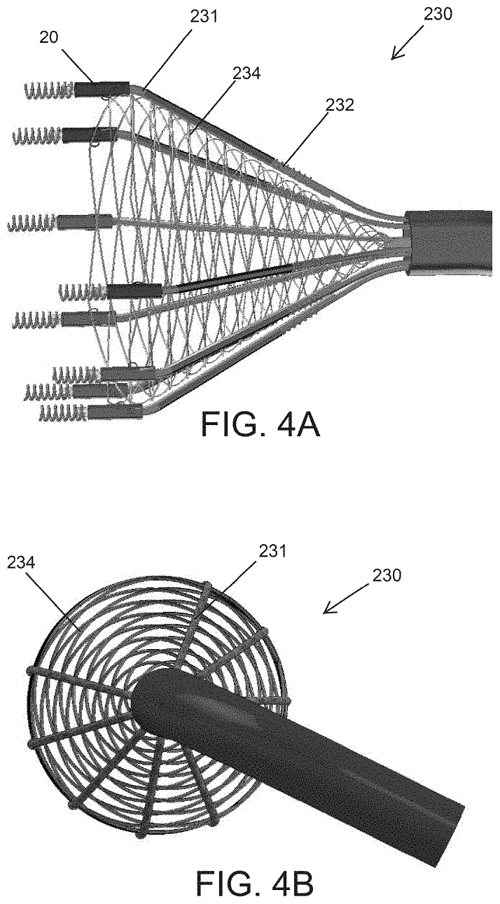

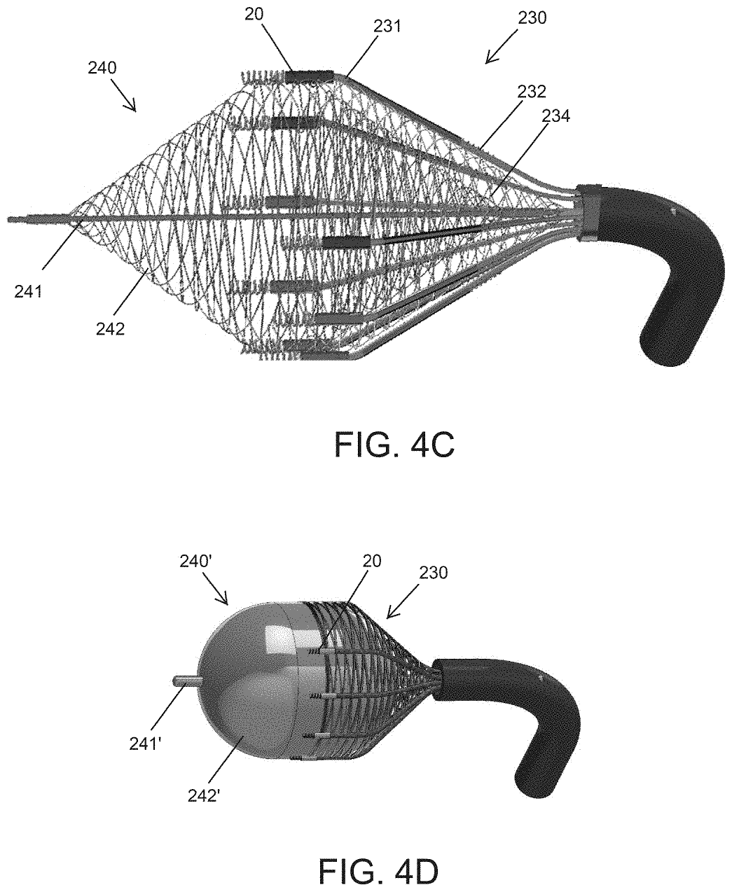

FIGS. 4A and 4B show a side view and rear view, respectively, of anchor support structure 230. As can be seen, the expandable frame 234 supports the array of support guides 231 through which the torque wires extend and which support the anchors 20. In some embodiments, the anchor support structure 230 supports the anchors 20 at anchors spaced radially with a uniform spacing. In other embodiments, the support frame can be configured to support the anchors at non-uniform spacing corresponding to the type of valve or the morphology of the valve annulus, as can be seen in FIG. 4B.

FIG. 4C shows the anchor support 230 disposed over the proximal portion of the expanded centering member 240, both of which include same or similar elements as those previously described. In this embodiment, the centering member 240 is a scaffold that allows blood flow therethrough, which advantageously allows for blood flow through the valve even during the centering procedure. FIG. 4D shows an alternative design in which the expandable centering member 240′ is a balloon 242′. This embodiment can still utilize a central shaft 241′ for alignment purposes during centering. A standard balloon could be used, in which case blood flow through the valve would be impeded during centering. Alternatively, a perfusion balloon could be used to allow flow of blood even with a balloon design.

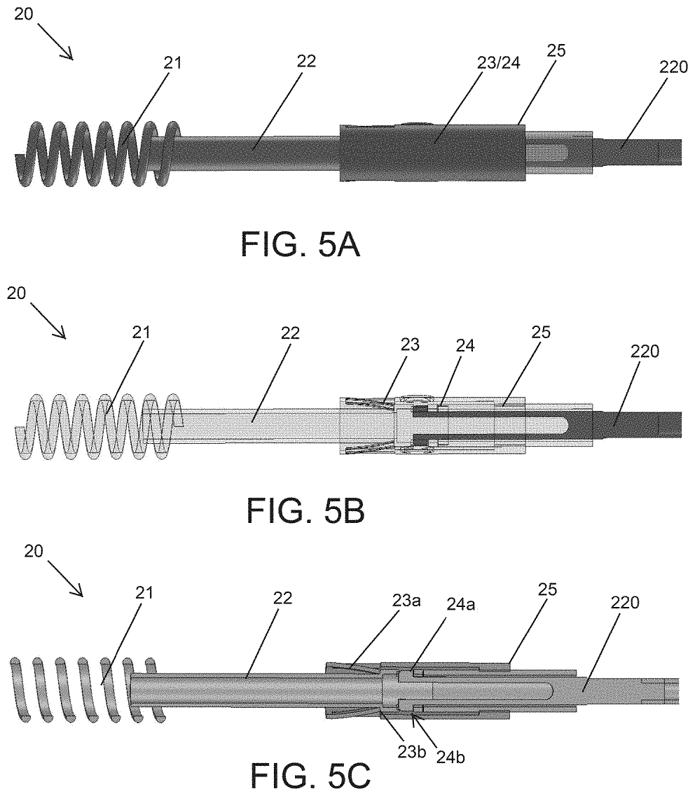

FIGS. 5A-5C show several views of screw anchors 20 in accordance with some embodiments. As described above, the anchors are analogous in function to the sutures in a conventional annuloplasty procedure. Each anchor 20 includes a distal penetrating tip 21 and a proximal shaft 22. In this embodiment, the distal tip is a helical screw that engages tissue and implants by rotation. Components of a ring locking mechanism 23 and a couple-release mechanism 24 are disposed on a proximal region of the shaft 22. The ring locking mechanism 23 secures a locking collar 25 attached to the annuloplasty ring (not shown) to the anchor shaft. The torque wire couple-release mechanism 24 couples the torque wire 220 to the proximal end of shaft 22 to facilitate driving of the screw anchor into tissue by torque of the torque wire and decouples the anchor from the torque wire when the ring is positioned and reformation of the valve annulus is determined to be sufficient.

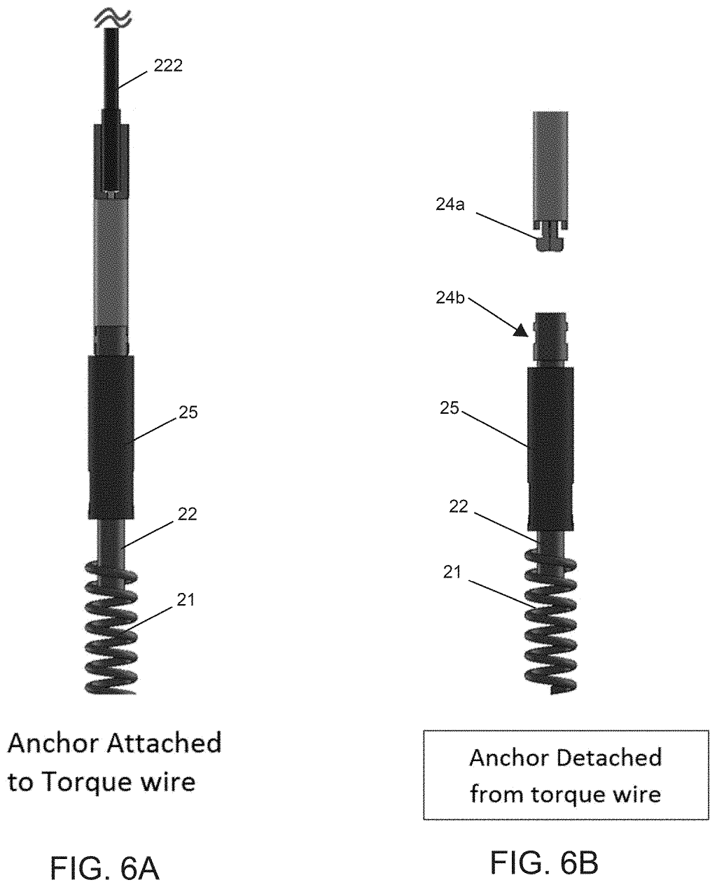

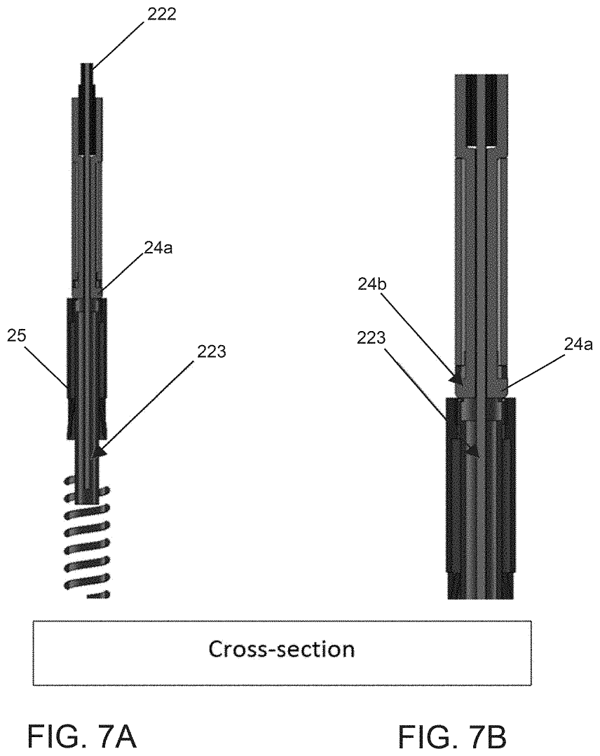

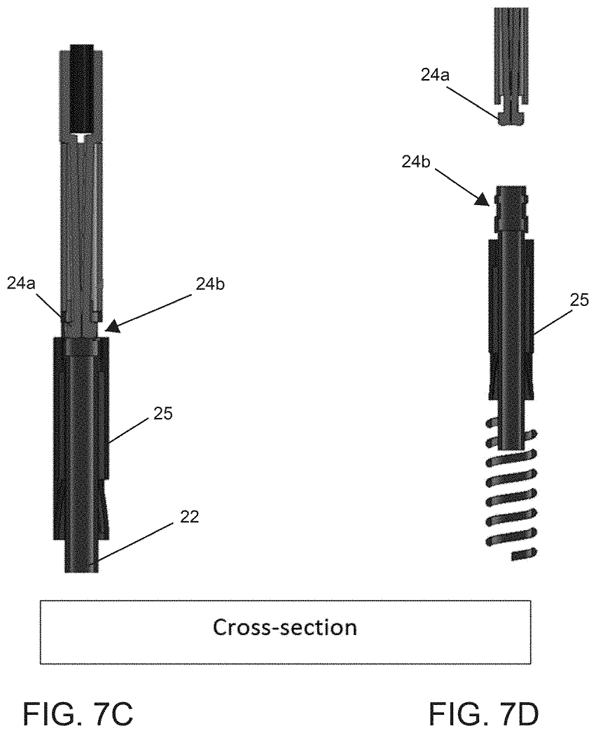

In the embodiment shown, the ring locking mechanism 23 includes a ridge 23a within the locking collar 25 that is biased inwardly in a proximal direction such that advancing the ring and locking collar 25 beyond a shoulder 23b on a proximal region of the anchor shaft 22, causes ridge 23a to deflect inwardly toward anchor shaft 22 and abut against the shoulder 23b, thereby locking the collar 25 and attached ring to the anchor. The couple-release mechanism 24 can includes a slot 24b at a proximal end of the anchor shaft 22 that receives a corresponding distal flange or ridge 24a on inwardly biased distal members of the torque wire so as to interlock and couple the torque wire with the anchor shaft. The operation of the torque wire couple-release mechanism 24 is further depicted in FIGS. 6A-6B and 7A-7D.

FIG. 6A shows the anchor shaft 22 attached to the torque wire 222 with locking collar 25 (ring not shown) locked to the anchor shaft. FIG. 6B shows the torque wire 222 detached from the anchor shaft 22, disengaged by the couple-release mechanism 24. As shown, the ridge 24a is disposed on inwardly biased distal members that deflect inwardly upon removal of an inner core wire so that ridge 24a disengaged from slot 24b along the proximal end of anchor shaft 22. FIGS. 7A-7B show cross-sectional views of the assembly before and after release of the torque wire 222 after the locking collar 25 with ring (not shown) has been secured to the anchor. As shown in FIGS. 7A-7B, central core wire 221 extends through torque tube 222 forcing the inwardly biased members apart so that distal ridge 24a extends laterally outward into the slot 24b of the anchor shaft 22, thereby locking torque wire 222 to the anchor. As shown in FIG. 7C, when core wire 221 is removed, the inwardly biased members of locking component 24a recover to their stress free state so that the members are drawn inward and ridge 24a is removed from slot 24b, thereby disengaging from the anchor shaft 22 to allow withdrawal of torque wire 222, as shown in FIG. 7D.

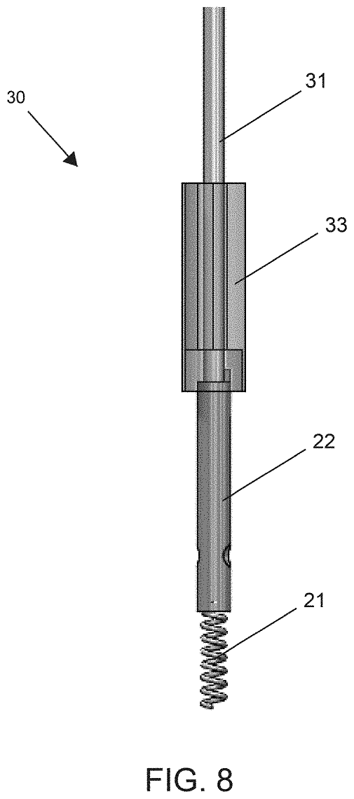

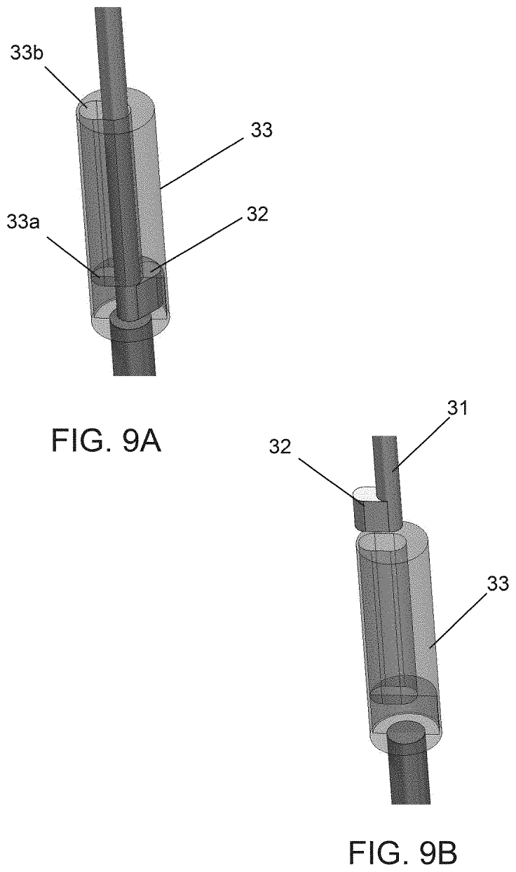

In another embodiment, the couple-release mechanism can include a rotating cam lock. As shown in the embodiments of FIGS. 8-11, the rotating cam lock 30 can include a cam lock 31 that interfaces with a locking sleeve 33 attached to the anchor shaft 22. As shown in the detail views of FIGS. 9A-9B, cam lock 31 includes a shaft and a distal cam 32 that can be positioned in a locked position (see FIG. 9A) during anchor delivery and deployment. As shown, the cam 32 is in a turned locked position within a corresponding shaped cavity 33a within the distal portion of the locking sleeve 33, which prevents the cam lock and attached torque tube from sliding out of the locking sleeve. After the annuloplasty ring is placed and secured to the anchors, the torque wires are released by twisting the cam lock 31. The cam lock 31 shaft can be rotated from their proximal end outside the patient, which rotates the cam 32 to align with a longitudinally extending slot 33b to allow cam 32 to be proximally retracted from the locking sleeve 33, thereby releasing the torque wires from the anchors.

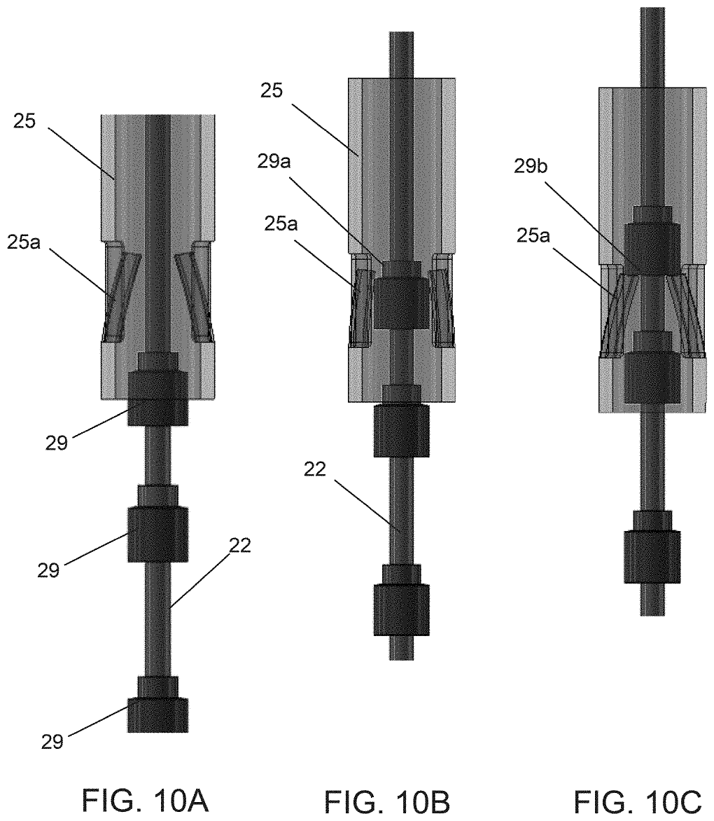

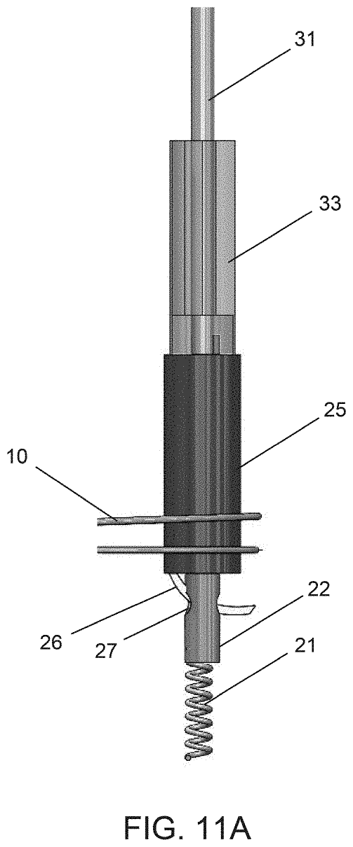

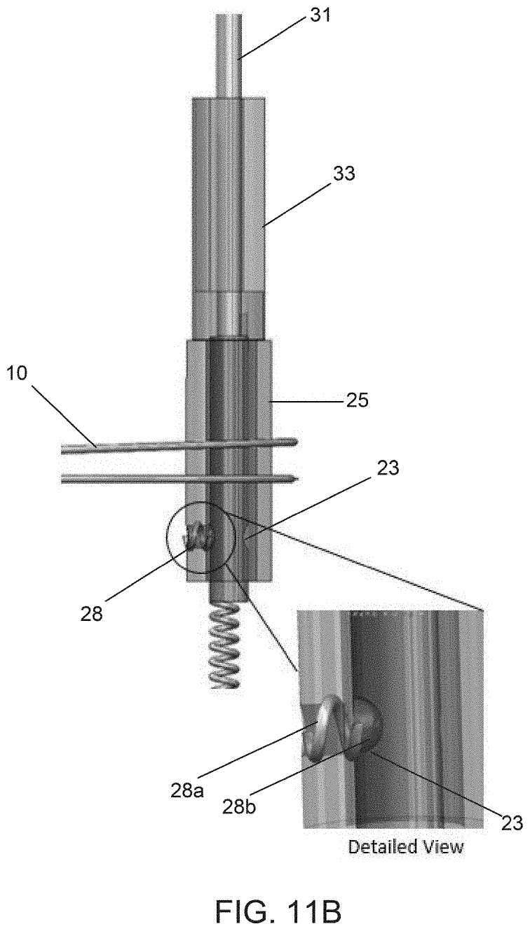

In another aspect, the ring locking mechanism can include a protruding element of a locking collar attached to the ring that interfaces with a hole, recess, or protruding feature of the anchor body or shaft. Examples of such mechanisms are shown in the embodiments in FIGS. 10-11B. In one embodiment, the ring coupling mechanism includes a hook coupling in which a hook or resiliently biased member on the annuloplasty ring or attached locking collar interface with a hole or recess on the anchor.

As shown in FIGS. 10A-10C, the anchor shaft 22 can include one or more hypotube features 29 that lock against one or more inwardly extending tabs 25a of the collars 25 inclined in the proximal direction. In this embodiment, the anchor includes a series of three hypotube features 29, which allows for adjustability, and the collar includes at least two inwardly extending tabs. As can be seen in FIG. 10A, each of the locking hypotube features has a tapered proximal end 29a, which allows the sleeve to be slid over the hypotube, thereby pushing the inwardly extending resilient tabs of the sleeve outward, as shown in FIG. 10B. Further advancement of the sleeve allows the inwardly extending tabs to resiliently deflect inward to their set position and lock against a distal flat end 29b of the hypotube, as shown in FIG. 10C. The inwardly extending tabs 25a can be formed of any suitable material, including the same material as the collar or a differing material. In some embodiments, the one or more tabs are integrally formed with the collar. In other embodiments, the one or more tabs are separately formed and coupled with the collar. In some embodiments, the one or more tabs are formed of Nitinol and are set in the inwardly extended positions. As shown, the ring can lock onto any of the three locking hypotube features. This configuration allows the ring to accommodate variations in anchor positioning and depth relative the ring/annulus.

As shown in FIG. 11A, the anchor shaft 22 is attached to a locking collar 25 which includes a distally extending hook 26 that extends through a hole 27 in the anchor shaft 22 when the ring 10 and attached collar 25 is advanced over the torque wires 222, thereby locking the ring to the anchor. In another embodiment, the ring coupling mechanism includes a locking collar with a spring-loaded ball that interfaces with a detent in the anchor body.

As shown in FIG. 11B, the locking collar 25 attached to the ring 10 includes a laterally extending, inwardly biased member 28 that interfaces with a hole or detent 23 within the anchor. As shown in the detail view, member 28 includes a spring 28a that biases a distal ball 28b inwardly so that when the collar is advanced over the anchor, the ball 28b is forced by spring 28a into detent 23, thereby locking the ring to the anchor, after which the torque wire can be detached as described above. While these examples are shown with the cam lock couple-release mechanism, it is appreciated that these ring coupling mechanisms could be used with various other embodiments as well.

In some embodiments, the couple-release mechanism can be configured such that engagement of ring locking mechanism actuates the torque wire couple-release mechanism to decouple the torque wire. For example, engagement of inwardly biased ridge 23a with the anchor shaft 22 can actuate a member that decouples coupling features 24a,24b to allow release of the torque wire. This design is advantageous as locking of the ring with the lock mechanism effects release of the torque wires. While a particular design of the lock mechanism and couple-release mechanism are shown and described above, it is appreciated that these mechanisms can include any interfacing components or any suitable connectors configured to provide the functionality noted above.

In this embodiment, the anchor tip and shaft are fabricated from stainless steel, although any suitable material can be used. The anchor can be formed of an integral component or can include multiple components attached together. Typically, the anchors are provided as described with the lock mechanism and couple-release mechanism attached thereto. While screw anchors are described herein, it is appreciated that any suitable type of anchor can be used including barbed anchors that are driven into tissue by applying an axial force from driving members connected to the anchor shaft. In this approach, the anchors can be deployed and removed in a similar manner, selecting any, all or any combination of anchors.

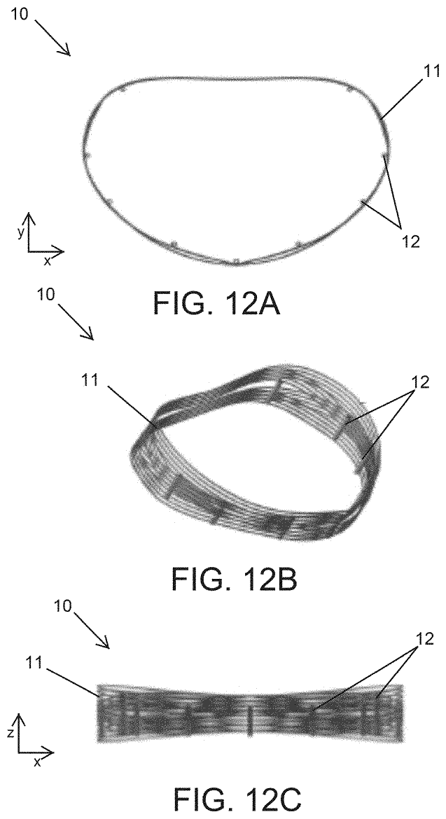

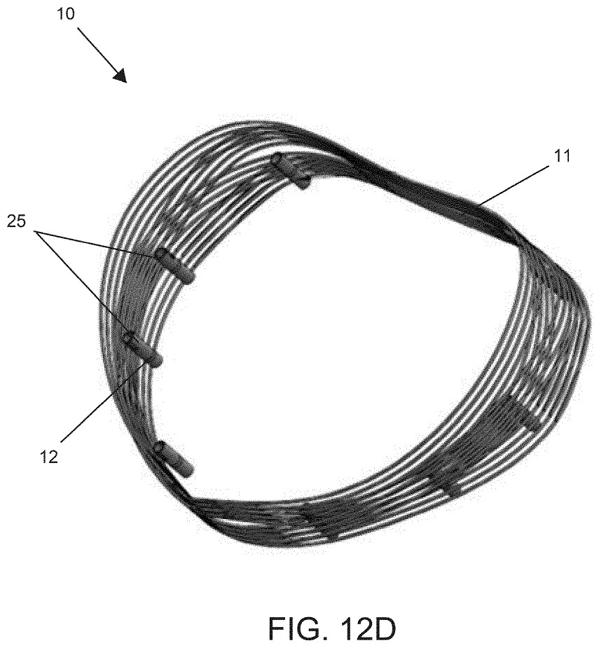

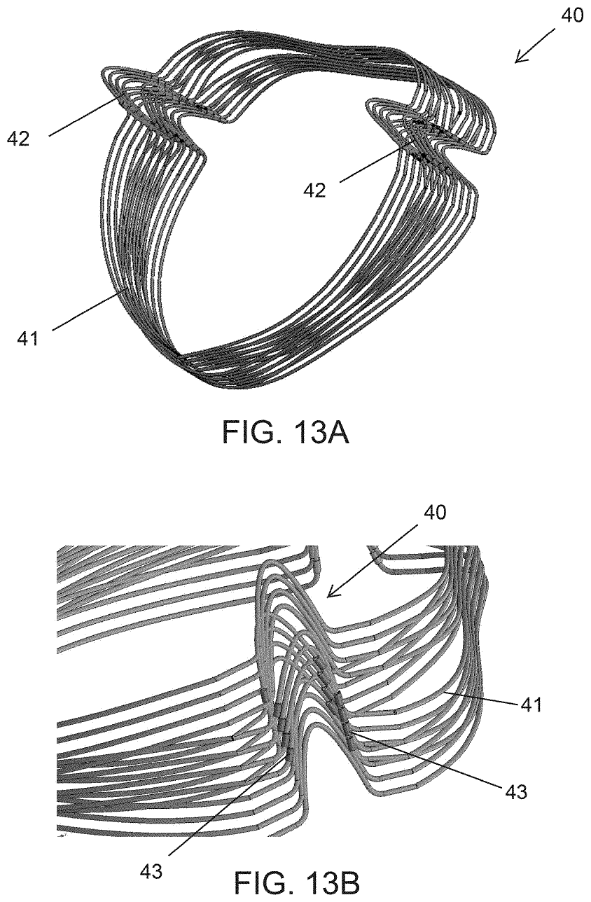

FIGS. 12A-12C show several views of an annuloplasty ring 10 in accordance with some embodiments. The ring 10 includes multiple concentric loops or rings 11 and a series of openings or eyelets 12 that receive the anchors to implant and secure the ring 11 against the valve annulus. In this embodiment, the annuloplasty ring is formed of a shape-memory alloy, such as Nitinol, and heat-set into three dimensional shape that mimics the healthy anatomical shape of the annulus. This allows the ring to be collapsed into a relatively small sized delivery catheter and to resume the desired shape when deployed from the catheter and secured to the anchors surrounding the valve annulus. Typically, the annuloplasty ring is semi-rigid. Advantageously, the three-dimensional design allows a variety of shapes and sizes to match the patient anatomy and specific characteristics of the mitral regurgitation in the patient, thereby providing a customized treatment approach. Evaluation of the patient pre-procedure with standard imaging techniques can be used to determine the shape and size ring for a given patient's anatomy. As shown in FIG. 12D, the ring 10 can include eyelets, each having a collar 25 to facilitate advancement of the ring over wires or cables. In this embodiment, the ring 10 includes eight collars at the eyelet locations, which are spaced non-uniformly at locations desired to anchor the ring along the valve. It is appreciated that the ring can include more or fewer collars at various other locations. The collar 25 can further include a ring locking feature, such as any of those described herein. In another aspect, the annuloplasty ring can be adjustable, for example as show in FIGS. 13A-13B described further below.

As shown, the annuloplasty ring 10 includes multiple concentric loops or rings that together form the ring structure. In some embodiments, the ring include any suitable number of loops, for example between 2 and 50, 5 and 30, or 10 and 20. The loops are generally of a similar 2D shape as each other, as can be seen in FIG. 6A, that corresponds to the desired 2D shape of the valve annulus. In this regard, the ring is similar to a shape of a conventional annuloplasty ring along two dimensions (x-y direction). However, the multiple loops can have differing shapes along the third dimension (z-direction), as can be seen from the side view in FIG. 6C. This 3D shape allows the annuloplasty ring to reform the valve annulus along an additional dimension, thereby better reforming the dilated valve annulus to a desired 3D shape to further improve coaptation of the leaflets of the valve. In one aspect, the annuloplasty ring designs can be optimized and evaluated for radial strength, ability to deploy and low profile.

In another aspect, the annuloplasty ring can include adjustable sections or portions that can be tightened or loosened to adjust the overall shape and/or size of the ring from outside the patient during deployment. In some embodiments, the function of the heart can be monitored during deployment and the ring adjusted accordingly until a desired heart valve function is achieved. In some embodiments, the ring includes v-shaped elements at specific locations that can be cinched tighter, as needed in order to reduce the size of the ring. As shown in FIGS. 13A-13B, the adjustable annuloplasty ring 40 includes multiple concentric wire loops 41 with two v-shaped elements 42. In the embodiment shown, the v-shaped elements 42 are located on opposite sides, along to major axis of the oval. This results in a reduction of the minor axis which corresponds to the septal-lateral direction on the valve, which is typically the most effective direction for mitral valve reduction. It is appreciated, however, that the adjustment portions could be located at various other locations and utilize various other constructions.

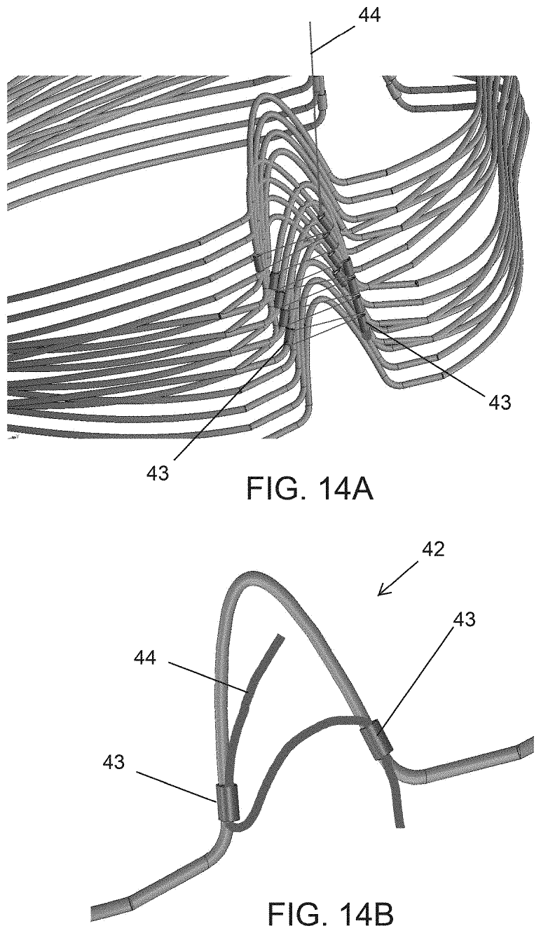

As shown in FIG. 13B, each wire of the v-shaped element includes a collar 43 on opposite sides. Collars 43 are fixed on the wider portions of the v-shape element and designed so that a cable can be passed through the collars. As shown in FIGS. 14A-14B, cable 43 is positioned through the multiple collars so that it is fixed on one collar and routed to span each of the v-shaped elements and extends outside of the of the patient so that the v-shaped portion can be tensioned/tightened by the clinician during deployment of the implant system. When the cable 43 is tensioned, the collars are brought closer together, reducing the dimension along the v-shaped element.

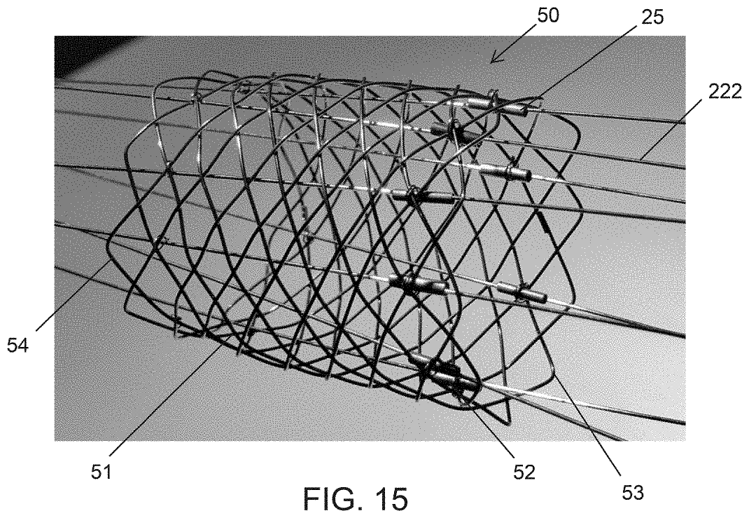

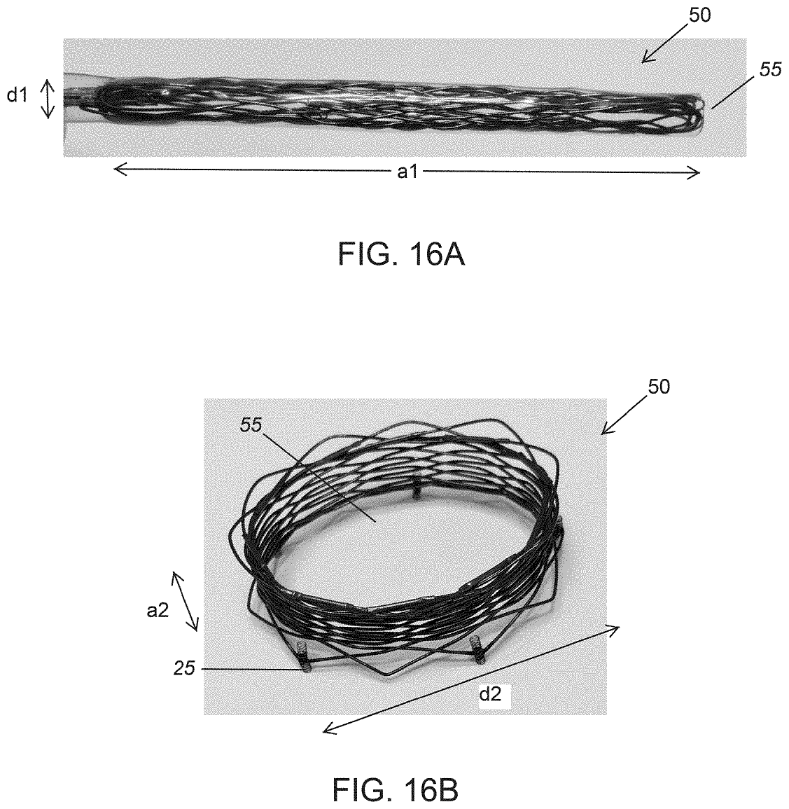

In another aspect, the annuloplasty ring can have a braided wire design that can be elongated and have a reduced diameter during delivery and then radially expanded to form the annuloplasty ring attached to the anchors. As shown in FIG. 15, the annuloplasty ring 50 is designed as an expandable scaffold formed of braided wire 51 that is interwoven about a central opening. In this embodiment, the wire 51 is a shape memory alloy, such as Nitinol. The scaffold includes eyelets 52 disposed near a distal portion of the scaffold, the eyelets having a locking collar 25, as described previously. Preferably, the scaffold has top end 54 and bottom end 53 that are each atraumatic, for example, without any exposed wire ends. As shown, the wire ends are connected to each other within the braid to form a continuous wire braid. In this embodiment, the top and bottom ends have a zig-zag design with peaks and valleys. In FIG. 15, the scaffold is shown being advanced along cable wires, midway between the delivery configuration, shown in FIG. 16A, and the deployed configuration, shown in FIG. 16B.

In the delivery configuration shown in FIG. 16A, the scaffold is axially elongated such that axial dimension a1 is larger than the diameter d1. As shown, the axial dimension is about 10 times as long as the diameter such that the scaffold resembles an elongated tubular shape along the longitudinal axis. The first diameter is sufficiently small to fit through a vascular access sheath, preferably a 18 French access sheath or smaller to allow delivery of the implant system to the heart valve through the femoral artery. The first axial dimension is typically between 2 cm and 10 cm.

In the deployed configuration shown in FIG. 16B, the scaffold is radially expanded and axially collapsed such that the diameter d2 is greater than the axial dimension a2. As shown, the average diameter is about five times greater than the axial dimension. When formed of a shape memory alloy, such as Nitinol, the scaffold is heat set into this deployed implantation configuration such that once delivered into the heart, the scaffold assumes this configuration. As shown, the scaffold resembles an oval shaped ring extending circumferentially about the central opening 55. Typically, the diameter d2 is within a range of 2 cm to 4 cm and suited for being secured around a heart valve, such as the mitral valve. The axial dimension a2 is relatively small, typically within a range of 0.5 cm to 3 cm.

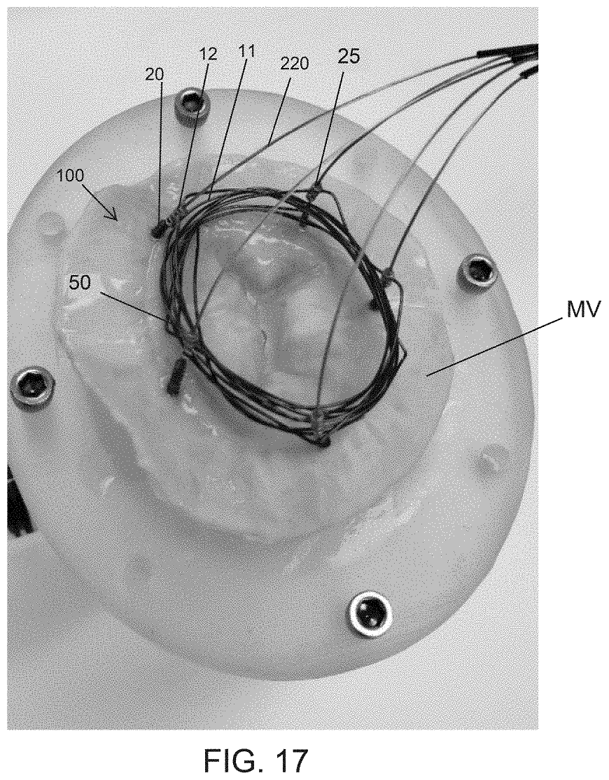

FIG. 17 shows an exemplary annuloplasty implant system 100 implanted on a model of a mitral valve annulus (MV) in accordance with some embodiments. In accordance with the embodiments described above, the implant system includes annuloplasty ring 50 and multiple screw anchors 20 implanted in tissue surrounding the MV. As can be seen, the torque wires 220 are still attached to the proximal end of the anchors 20 and the implant 50 has been advanced over the torque wires extending through the eyelets 12 and collars 25 and assumed the implantation configuration adjacent the annulus. The ring can then be locked to the anchor shafts while the torque wires 222 are decoupled from the anchors and removed leaving the implant in place. In some embodiments, the function of the valve can be assessed before the ring is locked into place so that adjustments can be made to the anchors or ring before decoupling the torque wires.

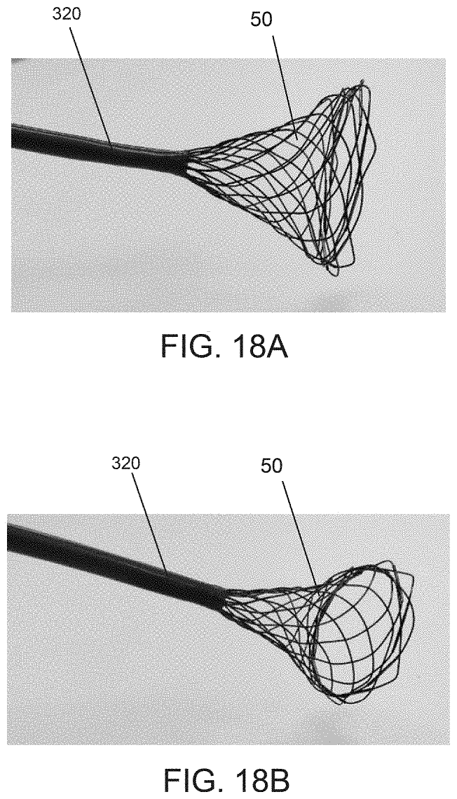

FIGS. 18A-18B shows the annuloplasty ring 50 being deployed from a ring deployment catheter. As can be seen, the annuloplasty ring can be constrained within a relatively small lumen of a catheter shaft 320 of the delivery catheter. The flexible braided scaffold design allows the entire ring to be axially elongated and radially collapsed and drawn into the catheter. The braided design has a mesh-like appearance, as shown in FIGS. 18A-18B, before being distally advanced and deployed to form the annuloplasty ring.

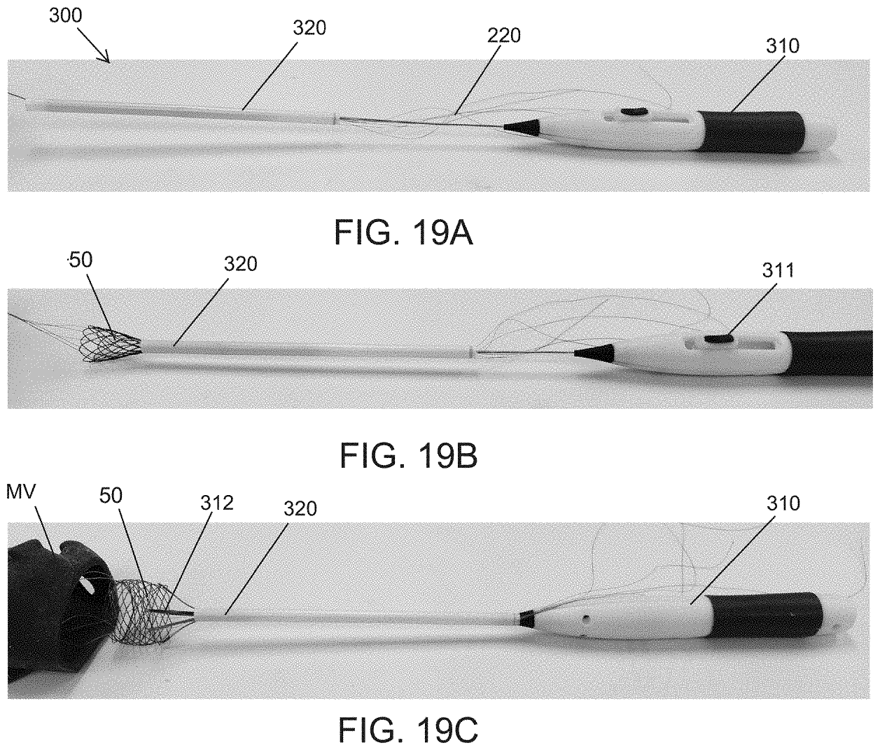

FIGS. 19A-19C show several views of an annuloplasty ring delivery catheter 300 in accordance with some embodiments. The delivery catheter 300 includes a proximal handle 310, an elongate flexible shaft 320, and an annuloplasty ring 10 constrained within a distal portion of the shaft. After removal of the anchor delivery catheter, the torque wires are left in place and the proximal ends of the torque wires are fed through the eyelets of the annuloplasty ring and then the ring is compressed and loaded into the shaft 320 with the torque wires 220 extending proximally from the shaft, as shown in FIG. 9A. The entire assembly is advanced over the torque wires to the mitral annulus. The ring can be deployed by proximal retraction of the shaft and/or by advancement of one or more pusher members 312 that engage the ring. The pusher members 312 extend to a control switch 311 on the handle. In this embodiment, the pusher elements are attached to the smaller catheter shaft which is attached to the handle. Advancement of the handle body will deploy the ring. Retraction of the handle body will pull the ring back into the larger shaft. The control switch on the handle disengages the pusher members from the ring and releases the ring from the catheter. Once released, the ring assumes its deployed configuration and can be attached to the anchors around the valve annulus, as described above.

As shown in FIG. 9C, pusher member 312 can include multiple arms that engage the ring to facilitate advancement and deployment of the ring adjacent the valve annulus. At this point, the shape and/or function of the reformed valve can be assessed by visualization techniques. If the physician determines the shape of the valve or valve performance is unsatisfactory, the ring can be removed by pulling the torque wires taut from the proximal end and drawing the ring within the sheath. The ring can then be withdrawn and adjusted or replaced as needed and the procedure repeated and re-assessed. Once the shape of the valve and/or valve function is satisfactory, the ring can be further advanced to secure the ring to the lock mechanism of the anchor shafts by the ring locking mechanism and decouple the torque wires from the anchors by the couple-release mechanism.

As shown, the pusher element comprises multiple arms that splay laterally outward and engage the most proximal loop of the prosthetic to allow axial movement of the pusher member to advance or retract the ring. The arms can be engaged with the loop by hooks, a coupling mechanism or any suitable releasable connector. In some embodiments, the pusher member can include one or more tubes disposed over one or more of the torque wires. While the ring delivery catheter is described as a separate catheter that is used after removal of the anchor delivery catheter, it is appreciated that the catheters can be combined within a single catheter in some embodiments.

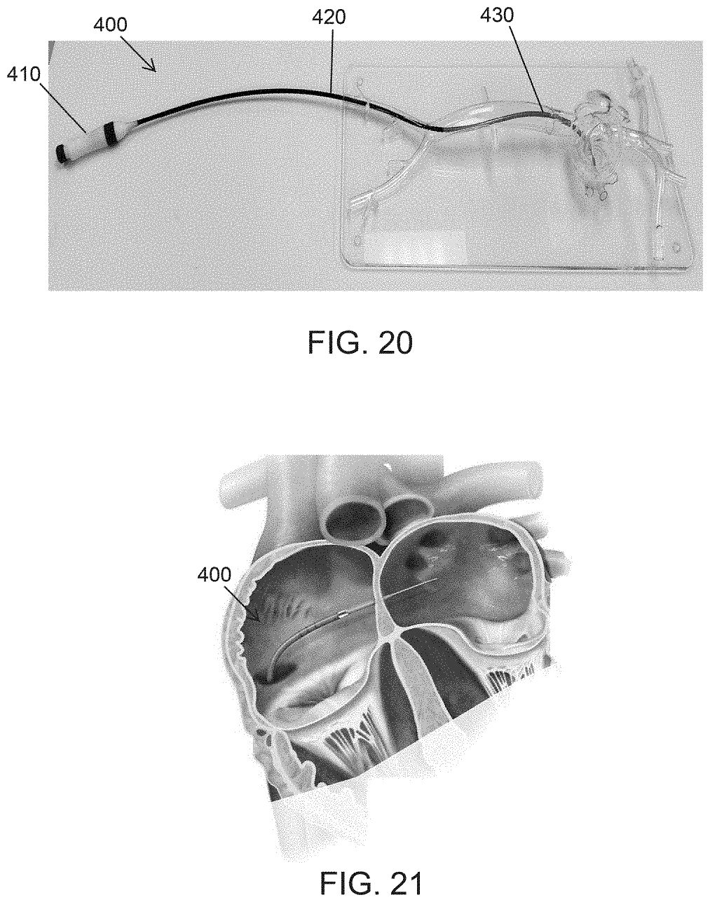

FIG. 20 shows an articulable access sheath 400 that can be advanced intravascularly to an atrium of the heart to provide access for the respective delivery catheters of the anchors and annuloplasty ring in accordance with some embodiments. The access sheath can include a proximal handle 410 with proximal access opening, an elongate flexible sheath body 420 and a flexible articulable distal region 430. In some embodiments, the access sheath is a deflectable 20F sheath to aid in delivery and positioning of the implant system. This access sheath allows the above-noted implantation procedure to be performed in a transfemoral-transseptal approach from a venous access site. The mitral valve can be accessed from the atrial side by a right to left atrial puncture. FIG. 21 shows the access sheath advanced through the septal wall and into the left atrium to provide access to mitral valve in the left atrium.

FIGS. 22A-22H show sequential views of an exemplary method of delivery and implantation of the anchors and annuloplasty implant system in accordance with some embodiments.

In FIG. 22A, the delivery catheter is advanced to the mitral valve from the atrial side. The assembly of the anchor support 230 and centering member 240 is then advanced so that the center shaft 241 of the centering basket enters the mitral valve, as shown in FIG. 22B. As shown, the assembly is positioned so that the center shaft of the centering assembly extends through the valve annulus into the ventricle, while the anchor support frame remains above the valve annulus in the atrium. The position of the assembly within the valve annulus can be confirmed by visualization techniques.

As shown in FIG. 22C, the centering member 240 is expanded within the valve annulus (for example by axial movement of a control switch on the proximal handle), thereby centering the assembly within the valve annulus. As can be seen, since the anchors 20 are supported further outside of the centering member, thereby positioning anchors surrounding the valve annulus. If needed, the anchor support 230 can be further advanced to ensure sufficient contact with surrounding tissues. As discussed previously, the anchor support can include spring portions that allow the anchors more leeway and conformability so that all anchors can suitably engage with surrounding tissue regardless of uneven contours of the tissues. Advantageously, the centering member can be a basket or scaffold to allow blood flow between the atrium and the ventricle even during the centering procedure.

As can be seen in FIG. 22D, the centering member has been contracted and axially retracted into the delivery catheter. Advantageously, this allows the valve to function while the physician continues the process of securing the anchors into the surrounding tissue. While the anchor support 230 supports the torque wires (not shown) and anchors in the proper position, the physician actuates the torque wires to drive the screw anchors into the surrounding tissue. As noted above, the physician can select any, all, or any combination of the screw anchors or can explant individual anchors as needed. Preferably, multiple anchors are deployed concurrently, which improves the ease of implantation and reduces the length of the overall procedure.

As shown in FIG. 22E, after the screw anchors 20 are satisfactorily implanted in the surrounding tissue, the anchor support can be withdrawn, along with the delivery catheter, leaving the torque wires in place extending through access sheath 400. The annuloplasty ring is then fed onto the proximal ends of the torque wires via the eyelets and loaded into the ring delivery catheter as described previously.

As shown in FIG. 22F, the annuloplasty ring is then advanced from the ring delivery catheter 300 over the torque wires 221. As can be seen in FIG. 12G, the ring can be further advanced from the catheter by a pusher member(s) 312 so that the scaffold emerges from the delivery sheath and assumes the deployed configuration and then is secured to the anchors adjacent the valve annulus. At this point, the shape of the reformed valve and/or valve function can be assessed, and if needed, the ring can be retracted and adjusted or replaced based on the assessment. Once the physician determined the shape of the reformed valve and/or valve function is suitable, the annuloplasty ring 10 is locked to the anchor shaft via a lock mechanism (for example, by further advancement of the ring) and the torque wires are decoupled from the anchor shafts. The ring delivery catheter and access sheath can then be removed, leaving the annuloplasty implant system in place, as shown in FIG. 22H.

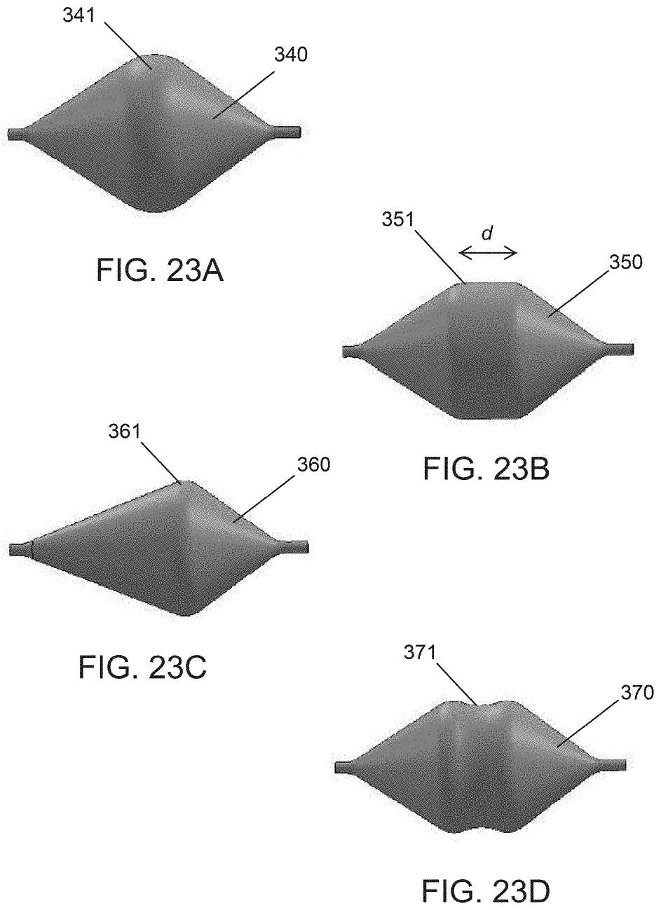

As can be understood by referring to FIG. 1B, the shape of the inner centering element is important for ensuring consistent within the mitral valve annulus A. The annulus is smaller in diameter compared to the atrium above and the left ventricle below the annulus such that the tissues form an hourglass shape with the annulus at the center. This natural shape of the annulus can make it difficult to reliably appose by engagement with an expandable centering structure. The un-modified shape of an expanded braided structure, as shown in FIG. 4C, has its largest diameter in the middle of the centering structure. In some embodiments, the greatest diameter of the centering portion is between 20 and 60, typically between 25 and 45 mm, As can be seen in FIG. 23A, the largest diameter portion 341 of expandable structure 341 is relatively narrow with respect to the angled proximal and distal portions. When expanded in the mitral valve space, the midpoint of the centering structure might shift to proximally or distally of the annulus. This variability in position relative to the annulus prevents the centering structure from reliably apposing and expanding the annulus, and may necessitate repeated repositioning. Accordingly, several different shapes can be utilized to reduce this variability, as shown in FIGS. 23B-23C.

In the embodiment of FIG. 23B, the centering structure 350 includes a long flat section 351 in the center that prevents bulging of the centering structure on either side of the annulus and better accommodates non-planar annulus shapes. In some embodiments, the long flattened section 351 extends a distance d, which can be between 5-50 mm, typically between 10-20 mm.

In some embodiments, the centering structure can include an enlarged region that is offset so that the structure deploys on one side of the annulus more consistently. For example, in the embodiment of FIG. 23C, the centering structure 360 includes an enlarged portion 361 that is off-center in the proximal direction. In still other embodiments, the centering structure can be defined in a shape to accommodate the annulus and automatically seat the braid within the annulus. For example, in the embodiment of FIG. 23D, the centering structure 370 includes an enlarged center portion 371 with a depression to receive the annulus within.

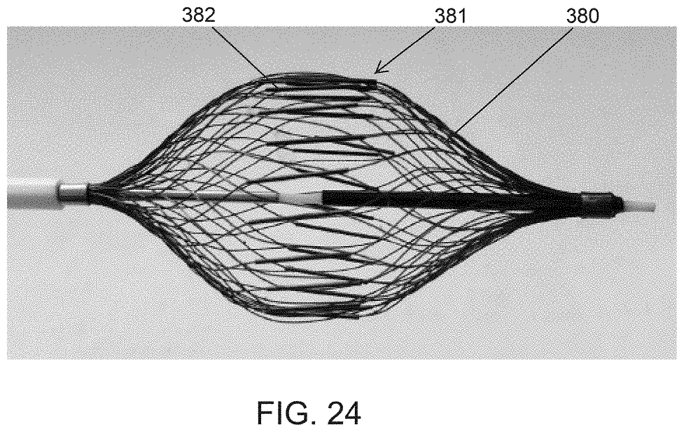

In some embodiments, such as that in FIG. 24, the centering structure is a braided wire-frame structure or basket 380 in which a series of hypotubes 382 are placed along the mid-section 381 to create a shape a flattened enlarged diameter portion 381 similar in shape to that in FIG. 23B. This creates a wider, flat section of the enlarged diameter portion 381 that more reliably apposes the annulus regardless of the angle relative to the annulus and non-planar shape of the annulus.

In another aspect, the anchor delivery catheter can include additional features to improve conformance with the annulus upon initial placement of the anchors about the annulus. When the inner centering structure is expanded within the mitral valve and the anchor delivery structure is advanced toward the annulus, the anchor housing and anchors need to conform to the annulus. All of the anchor housings should be in good contact with the annulus which can be a challenge given that the catheter may not approach the annulus at a perpendicular angle. While in some embodiments, the clinician can adjust advancement of individual anchor to conform to the annulus, it is desirable to improve conformability in a manner so that the anchors self-center and self-conform more reliably to the annulus. Therefore, to improve self-centering and self-conformance, the anchor delivery catheter can further include a flexible support band that supports the anchors about a central longitudinal axis and can accommodate various differing approach angles so that the anchors better conform to the surrounding annulus.

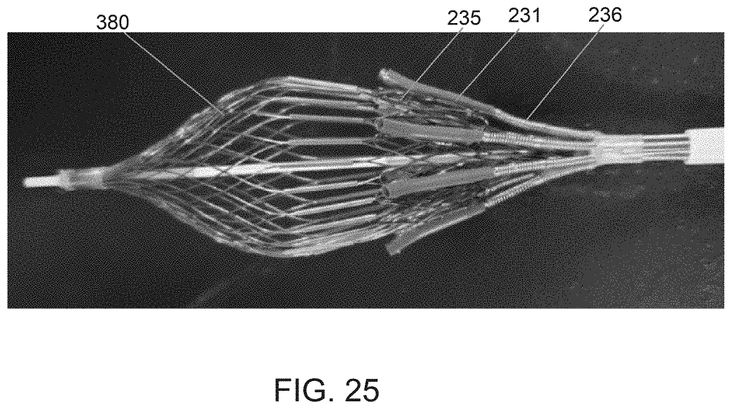

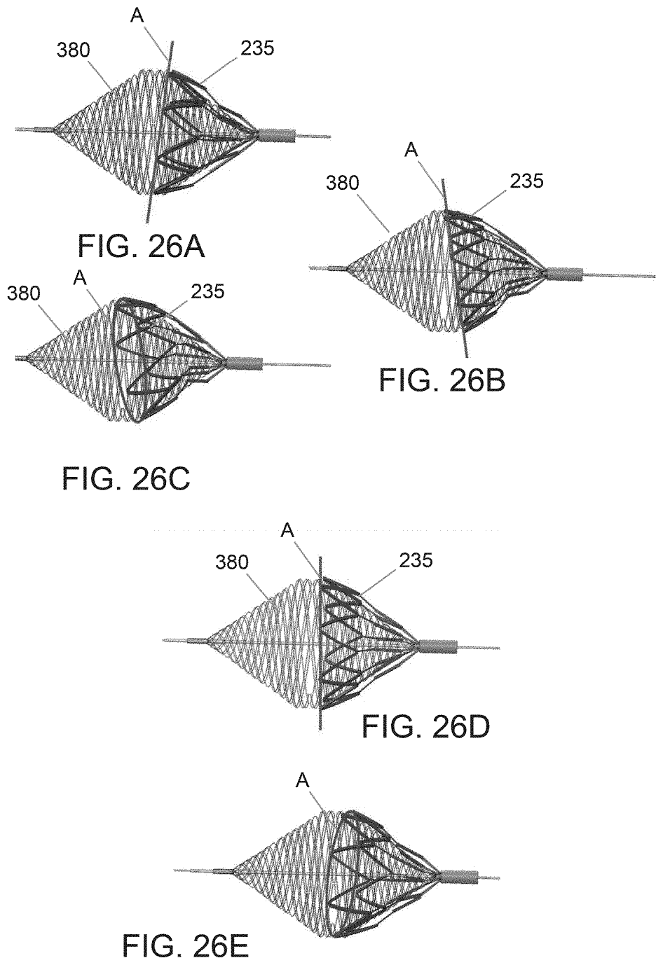

FIG. 25 shows an exemplary embodiment of an anchor delivery catheter having such a flexible support band 235. In this embodiment, the anchor housings are connected to the support band 235 which is connected via compression springs 236 to the proximal end of the centering structure. In some embodiments, the support band is connected to the anchor support, while in other embodiments, the support band can move separately from the anchor support. As shown, the support band is a flexible, expandable frame that supports multiple tubular supports 231 through which the anchors extend. The support band and tubular supports are proximally supported by multiple corresponding spring tubes 236. Thus, while the anchors are splayed outward by expansion of the inner centering structure, the anchor support band allows the anchors to conform to the annulus regardless of the approach angle, as shown in FIGS. 26A-26E.

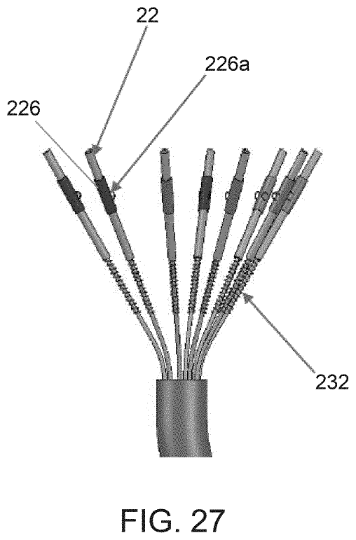

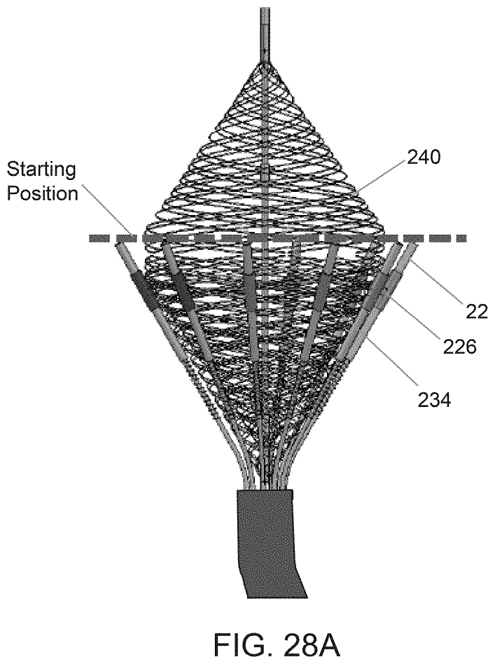

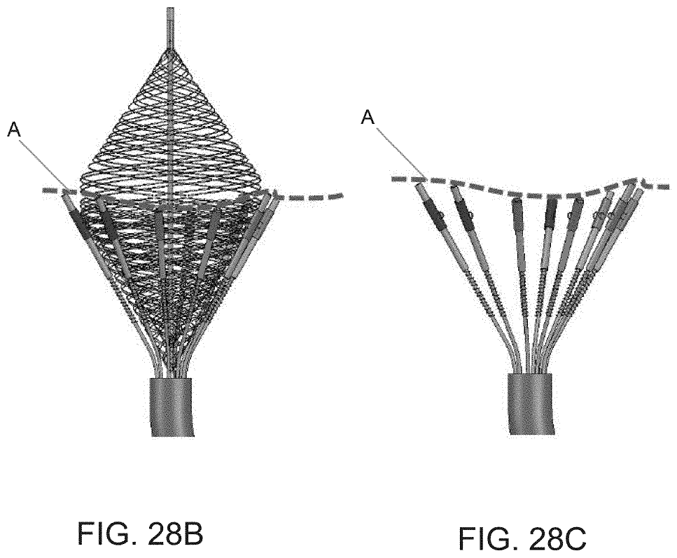

Another such feature utilizes slidable anchors and proximal springs that bias the anchors distally so as to engage and conform to the annulus tissues. In the embodiment of FIG. 27, each anchor housing 22 can slide through a sleeve 226. Each individual anchor housing 22 moves along its longitudinal axis and slides through sleeve 226 that is connected to an inner expandable anchor support structure that splays that anchors outward, or alternatively can be disposed on the centering structure. Each sleeve can be coupled to an inner expandable structure by a loop 226a, or by any suitable means. Springs 232 at the proximal end of the anchor housing 22 can be compressed so the anchor housings 22 are biased distally so as to conform to the annulus when the catheter is advanced toward the annulus A, as shown in FIGS. 28A-28C. FIG. 28A shows the starting position with all the anchors projected distally by the springs to a common plane. FIGS. 28B and 28C show the delivery catheter advanced distally to an irregular plane of annulus A, where select anchor housings 22 have slid through sleeves so as to better conform to the irregular contours of the annulus.

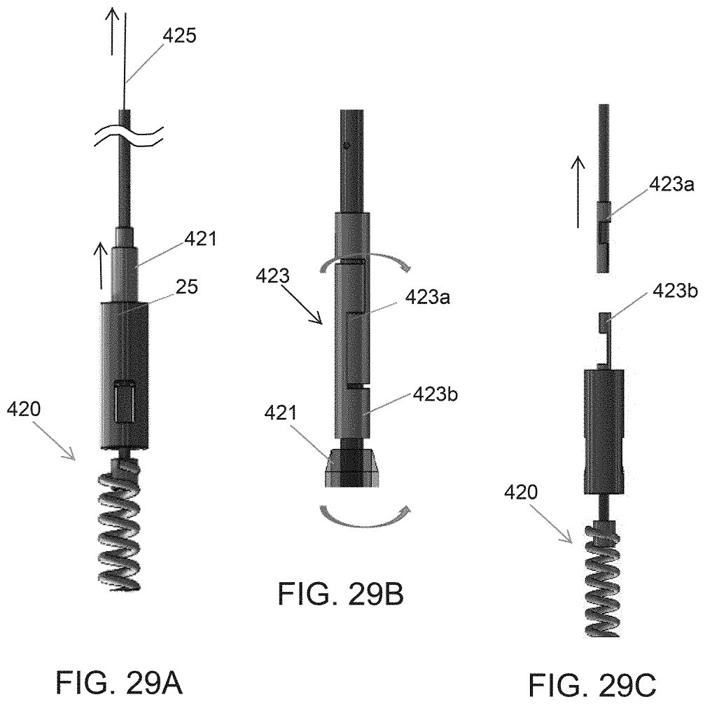

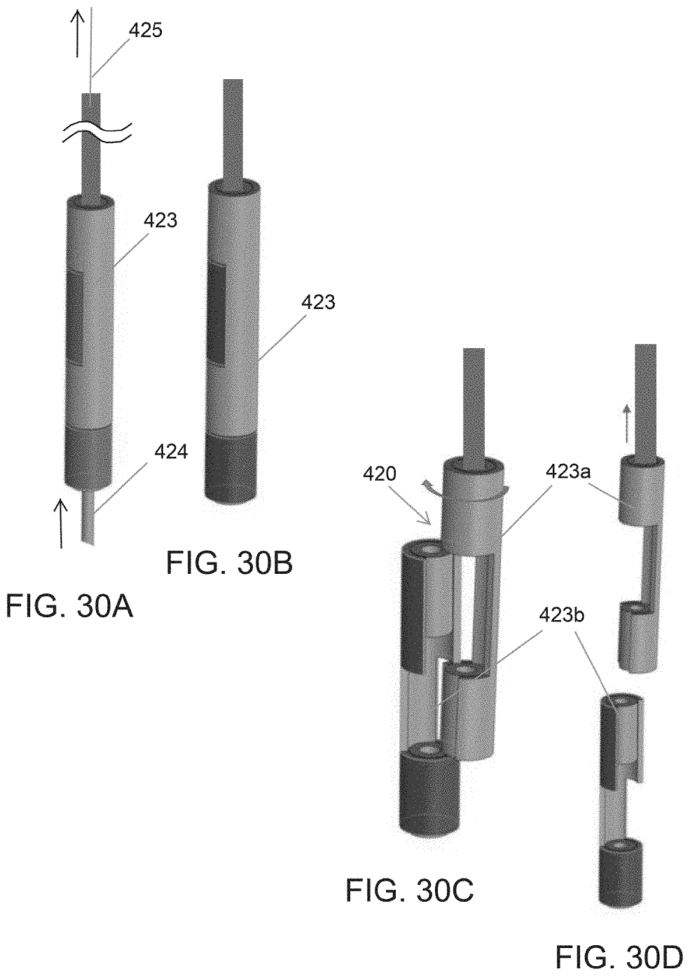

In another aspect, the anchor delivery catheter can include various other anchor release mechanisms than those described previously. In some embodiments, the anchor housing and torque wire are attached by interlocking pieces that are held together during delivery and ring locking by a coupler (e.g. sleeve, through-wire). In some embodiments, the coupler is removable or retractable so that the interlocking components can separate and the torque wire cables can be removed from the anchor bodies, while the anchor and ring remains locked on the annulus. Examples of such anchor release mechanisms are shown in FIGS. 29A-29C and 30A-30D.

In the embodiment of FIGS. 29A-29C, the interlocking components 423 include component 423a attached to the distal end of the torque cable and component 423b attached to a proximal end of the anchor shaft. The interlocking components assume a cylindrical shape when held together by an outer sleeve coupler 421. FIG. 29A shows the anchor 420 after locking of the ring collar 25 (ring is omitted for clarity). The outer sleeve 421 prevents interlocking components 423a, 423b from separating so that the torque wire and anchor housing remain securely coupled. After the outer ring is locked, the outer sleeve 421 is then withdrawn by retracting a pull wire 425 that runs through the inner lumen of the torque cable. As shown in FIG. 29B, the outer sleeve coupler 421 has been removed and the torque wire is torqued, which causes component 423a to rotate and separate from component 423b. The torque cable with component 423a can then be removed from the body, while the anchor 420 and locked ring remain secured to the annulus.

In the embodiment shown in FIGS. 30A-30D, the interlocking components 423, similar to those in FIG. 29A, include component 423a attached to the distal end of the torque cable and component 423b attached to a proximal end of the anchor shaft. The components are held together by an inner throughwire coupler 424 which extends through both components during anchor delivery and locking of the ring. After locking of the ring, the inner wire 424 is withdrawn, either by proximally retracting directly or by use of a pull wire 425 that runs through the inner lumen of cable, as shown in FIG. 30A. After throughwire 424 is removed, as shown in FIG. 30B, the torque cable is then torqued which separates components 423a, as shown in FIG. 30C, and the torque cable is then removed, as shown in FIG. 30D.

In the foregoing specification, the invention is described with reference to specific embodiments thereof, but those skilled in the art will recognize that the invention is not limited thereto. Various features, embodiments and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art. Each of the references cited herein are incorporated herein by reference for all purposes.

1. An anchor delivery system for delivery of anchors for a heart valve implant, the delivery system comprising:
a delivery catheter configured to extend from outside the patient to within the heart of a patient;
a plurality of anchors disposed within a distal portion of the delivery catheter, each anchor comprising:
a shaft extending between proximal and distal ends;
a distal penetrating tip disposed at the distal end;
a ring locking feature disposed along the shaft at or near the proximal end for locking the ring by a ring locking mechanism;
a torque wire couple-release feature disposed along the shaft at or near the proximal end and configured for decoupling a torque wire coupled with the shaft by a torque wire couple-release mechanism; and
a plurality of torque wires coupled to the respective shafts of the plurality of anchors via the couple-release mechanisms by corresponding couple-release features to allow for simultaneous deployment; and
a proximal handle of the catheter that controls actuation of the torque wires during anchor delivery.

2. The anchor delivery system of claim 1, wherein the torque wire couple-release mechanism comprises a first interlocking component on a distal end of a respective torque wire and a second interlocking component on a proximal end of an anchor shaft of a corresponding anchor, wherein the first and second interlocking components interlock to facilitate delivery and deployment of the anchors and are selectively releasable to allow release of the anchor and removal of the torque wire.

3. The anchor delivery system of claim 2, wherein the torque wire couple-release mechanism further comprises a coupler that couple the first and second interlocking components together and is selectively removable to separate the first and second interlocking components.

4. The anchor delivery system of claim 2, wherein the first and second interlocking components include interleaving portions extending partly in a circumferential direction such that, when coupled together, the interlocking components define a substantially cylindrical shape.

5. The anchor delivery system of claim 3, wherein the coupler is an outer sleeve disposed around the first and second interlocking components thereby securing them in an interlocked position, wherein the outer sleeve is configured to be selectively withdrawn from a proximal end of the delivery catheter.

6. The anchor delivery system of claim 3, wherein the coupler is a through-wire that extends through the first and second interlocking components in the locked position, wherein the through-wire is configured to be selectively withdrawn from a proximal end of the delivery catheter.

7. The anchor delivery system of claim 3, wherein the coupler is configured to selectively withdrawn by a pull wire extending through the respective torque wire.

8. The anchor delivery system of claim 1, wherein the torque wire couple-release mechanism comprises a laterally extending ridge disposed on one or more distal members that are inwardly biased and pushed outward by an inner core wire extending through the torque wire and between the distal members such that the ridge protrudes into a slot of the anchor, thereby coupling the torque wire to the anchors.

9. The anchor delivery system of claim 1, wherein the torque wire couple-release mechanism comprises a cam lock having a rotatable cam shaft with a distal cam that interfaces within a locking sleeve attached to the anchor body such that rotation of the cam lock moves the cam between a locked configuration to an unlocked configuration.

10. The anchor delivery system of claim 9, wherein
in the locked configuration, the cam is turned into a corresponding cavity in the locking sleeve and
in the unlocked configuration, the cam is turned into a longitudinally extending slot from which the cam can be longitudinally slid and withdrawn from the locking sleeve.

11. The anchor delivery system of claim 1, wherein the ring locking feature comprises any of:
shoulder or flange having a distal facing surface that abuts against a corresponding ring locking feature of the implant during implantation; and
a hole, detent or recess configured to receive a corresponding protruding locking feature of the implant during implantation.

12. The anchor delivery system of claim 11, wherein the ring locking feature comprises a hypotube on the anchor shaft, the hypotube having a tapered proximal portion to facilitate passage of one or more tabs of a collar of the ring thereon and a distal facing surface to abut against the one or more tabs of the collar to lock the ring to the anchor.

13. The anchor delivery system of claim 11, wherein the anchor shaft comprises a series of ring locking features so as to be adjustable.

14. The anchor delivery system of claim 1, wherein the ring locking mechanism comprises the ring locking features of the anchor and implant.

15. The anchor delivery system of claim 1, wherein the implant comprises an annuloplasty ring.

16. The anchor delivery system of claim 1, wherein each ring locking mechanism and torque wire couple-release mechanism are configured such that actuation of the ring locking mechanism with the heart valve implant effects actuation of the torque wire couple-release mechanism thereby decoupling the respective shaft and torque wire.

17. The anchor delivery system of claim 1, wherein the couple-release mechanism is located proximally of the lock mechanism on the anchor shaft.

18. The anchor delivery system of claim 1, wherein the lock mechanism comprises a resilient ramped surface in a proximal direction, wherein the ramped surface extends from an inside of a collar disposed on the shaft.

19. The anchor delivery system of claim 1, wherein the couple-release mechanism comprises a protruding feature that engages a corresponding protruding feature at or near a distal end of the torque wire, thereby coupling the torque wire with the shaft when the corresponding features are engaged.

20. The anchor delivery system of claim 1, wherein the couple-release mechanism is configured such that the engaged protruding features disengage when the resilient ramped surface of the lock mechanism is engaged with the ring, thereby decoupling the torque wire.

21. The anchor delivery system of claim 1, wherein the proximal handle includes a plurality of torque mechanisms configured to torque each torque wire, and a manually rotatable actuator to effect torqueing of the torque wires with the torque mechanisms.

22. The anchor delivery system of claim 1, wherein the proximal handle comprises a selector feature for each torque wire to allow a user to select any, all or any combination of torque wires for actuation to allow selective driving of any, all or any combination of anchors.

23. The anchor delivery system of claim 1, wherein the proximal handle is further configured to allow any, all, or any combination of torque wires to be selected and driven in reverse to allow removal of one or more selected implanted anchors.

24. The anchor delivery system of claim 1, further comprising:
an expandable anchor support supporting the plurality of anchors to facilitate positioning of the plurality of anchors about the valve annulus; and
an expandable centering member disposed at least partly within the expandable support frame.

25. The anchor delivery system of claim 24, wherein the expandable anchor support a plurality of guide tubes that support the plurality of anchors at distal ends thereof, wherein the torque wires extend through the guide tubes to allow driving of the plurality of anchors into tissue while supported by the guide tubes.

26. The anchor delivery system of claim 25, wherein the guide tubes are splayed laterally outward along distal portions thereof by an expandable scaffold of the anchor support.

27. The anchor delivery system of claim 24, wherein the expandable anchor is configured to support the anchors spaced laterally outward from the expandable centering structure.

28. The anchor delivery system of claim 24, wherein the expandable anchor support is configured to support the anchors spaced radially with a uniform spacing.

29. The anchor delivery system of claim 24, wherein the expandable support frame is configured to support the anchors spaced radially in a non-uniform spacing corresponding to the morphology of the valve annulus.

30. The anchor delivery system of claim 24, wherein the expandable anchor support includes spring portions along the guide tubes proximal of the anchors to allows the anchor support and anchors to be conformable during delivery and allows for uniform anchor and tissue interaction before deployment.

31. The anchor delivery system of claim 24, wherein the expandable anchor support defines a support band extending about a longitudinal axis, the support band being supported by the spring portions to accommodate varying approach angles when the anchors are advanced against the annulus.

32. The anchor delivery system of claim 24, wherein the centering member has a greatest diameter along a flattened enlarged region near the center to facilitate apposition with the annulus.

33. The anchor delivery system of claim 32, wherein the flattened enlarged region extends a distance of about 10-20 mm.

34. The anchor delivery system of claim 24, wherein the centering member is an expandable scaffold or basket that allows flow of blood therethrough.

35. The anchor delivery system of claim 24, wherein the expandable basket includes a plurality of hypotubes extending in the longitudinal direction so as to define a flattened enlarged region.

36. The anchor delivery system of claim 24, wherein the centering structure is a balloon.

37. The anchor delivery system of claim 24, wherein the centering member is independently expandable and axial movable relative the anchor support.

38. The anchor delivery system of claim 24, wherein the centering member has a greatest diameter along a region that is offset from a center to provide control deployment of the region either proximal or distal of the annulus.

39. The anchor delivery system of claim 24, wherein the centering member has a greatest diameter along central region, wherein the central region further includes a depression in the center so that the central region has an hourglass shape to accommodate the annulus within the depression.

40.-62. (canceled)
